(19) United States
(12) Patent Application Publication
(10) Pub. No.: US 2024/0099214 A1
(43) Pub. Date: Mar. 28, 2024

(54) PLANT DEFENCE GENE EXPRESSION

(71) Applicants: UPL Corporation Limited, Port Louis (MU); UPL Europe LTD, Warrington (GB)

(72) Inventor: Alberto Albertini, San Carlo di Cesena (IT)

(21) Appl. No.: 18/263,939

(22) PCT Filed: Feb. 9, 2022
(86) PCT No.: PCT/GB2022/050342

§ 371 Date: Aug. 2, 2023

(30) Foreign Application Priority Data
Feb. 10, 2021 (GB) 2101803.1

(51) Int. Cl. A01H 1/00; A01N 43/16

(52) U.S. Cl. CPC A01H 1/126; A01N 43/16

(57) ABSTRACT

The present invention relates to management of plant defenses and tolerance to insect and fungal vectored pathogens such as viruses and disease resistance management by applying laminarin or compositions comprising laminarin to a plant. Application of laminarin modulates the expression of plant defence genes in the target plant.

### TECHNICAL FIELD

The present invention relates to plant defenses and tolerance to insect and fungal vectored pathogens and disease resistance management using laminarin based compositions. Particularly, the invention relates to use and application of laminarin composition for increasing plant defenses and tolerance to insect and fungal vectored pathogens and pest/disease resistance and defense mechanism of the plant. More particularly, the invention relates to modulation of plant defence genes by laminarin based composition.

### BACKGROUND

Viruses belonging to the genus Orthotospovirus, Potyvirus, Caulimovirus, Cucumovirus, Tobamovirus and other genera infect a broad range of plant species including horticultural crops, impacting agriculture in tropical and subtropical areas globally. Annual losses due to these are estimated to be over $1 billion worldwide. Among these viruses, Tomato Spotted Wilt Virus (TSWV), Potato Virus Y (PVY), Cauliflower mosaic virus (CaMV), Cucumber mosaic virus (CMV), Tobacco mosaic virus (TMV) are amongst the most widely occurring and economically important with a host range of more than 1000 plant species. Crops that are affected by these viruses include tomato, bean, lettuce, peanut (groundnut), pepper, potato, tobacco, cauliflower and numerous ornamental species.

Plants react to viral pathogen by activating two main elaborate defense mechanisms. Symptoms are the macroscopic and final evidence of the infection. The first is the basal defense, based on the actions of the basal immune system. This system can be activated by the so-called elicitors, term widely used to indicate a diverse group of structurally non-related compounds that act as signal molecules for plants in danger. Elicitors specifically recognized by the plant are bacterial flagellins, lipopolysaccharides or elongation factors, and fungal chitin or heptaglucosides, viral proteins. These are referred to as pathogen-associated molecular patterns (PAMPs), or, more recently, as microbe-associated molecular patterns (MAMPs), because nonpathogenic microorganisms also have PAMPs. The second mechanism is based on the actions of the adaptive immune system, which is composed of resistance (R) genes that can specifically recognize host proteins. These are coded by the pathogen Avr genes and they confer a resistant phenotype to the plant, as postulated by the gene for gene theory. The elicited status leads the plant to cause overexpression of a set of defense-genes: once the defence-genes are activated the plants become less susceptible.

With the overuse of plant pesticidal agents, chemicals and damage caused thereby using such reparative measures, it has become a need of the hour to find preventive and curative solutions before the damage is done. Such an aim has propelled extensive research into innovative ways and novel substances that can fulfil the purpose and strengthen the plant, thus shielding it from damage by pests and diseases. As a result, strengthening plants' natural defence mechanism is one such strategy. A number of natural substances are being profiled and studied to understand the effects they exert. Laminarin, a natural substance abundantly present in seaweeds, is one such subject.

Although numerous research has been conducted in determining the effect of laminarin on plant defence mechanisms and disease resistance, there is a dearth of material reflecting on the mechanisms and underlying principles leading to the external outcomes and manifestations of laminarin's effect which would be a useful tool in optimizing the amount of chemical used and its corresponding effect. Thus, it was the need of the hour to find solutions to strengthening plant defenses and tolerance to insect and fungal vectored pathogens effecting at the genetic level.

Thus, it was an objective of the present invention to provide a solution for strengthening plant defenses and tolerance to insect and fungal vectored pathogens and pest/disease resistance at molecular and genetic level. It was an object of the present invention to provide a composition for preventive damage control instead of a curative reparative solution for plants. It was yet another objective of the present invention to characterize the molecular events leading to plant defenses and tolerance to insect and fungal vectored pathogens.

### SUMMARY

The invention therefore provides a method of increasing plant immunity, plant defenses and tolerance to insect and fungal vectored pathogens, increasing plant disease or pest resistance, protecting plants from disease, or improving plant disease management, by modulating gene expression of plant defence genes, said method comprising applying a composition comprising laminarin to said plant.

The invention also provides the use of laminarin to increase plant immunity, plant defenses and tolerance to insect and fungal vectored pathogens, increase plant disease or pest resistance, protect plants from disease, or improve plant disease management by modulating gene expression of plant defence genes by a method according to any one of the preceding claims.

The invention also provides a composition comprising laminarin for increasing plant immunity, plant defenses and tolerance to insect and fungal vectored pathogens, increasing plant disease or pest resistance, protecting plants from disease, or improving plant disease management, by modulating gene expression of plant defence genes.

### BRIEF DESCRIPTION OF THE DRAWINGS

FIG. 1—Gene expression compared to water treatment after 12 hr from application.

FIG. 2—Gene expression compared to water treatment after 24 hr from application

FIG. 3—Gene expression compared to water treatment after 48 hr from application.

FIG. 4—Gene expression compared to water treatment after 72 hr from application

### DETAILED DESCRIPTION OF THE EMBODIMENTS

Vaccination is an established technique of generating an immune response before the disease strikes in animals and humans. However, this technique has rarely been practised in plants. Rather, curative measures are more implemented in plants than preventive measures. With profound use of agrochemicals and pesticides and consequential damage to the environment, phytotoxicity in plant and resistance in pests, a technique wherein the defenses and tolerance to insect and fungal vectored pathogens of plants is strengthened by a means similar to vaccination could be more useful and impactful in the future. The inventors of the present invention found an unexpected result on these very lines on applying laminarin to the plants. To their surprise, the inventors of this invention found that laminarin was able to effectively modulate the expression of plant genes involved in defence mechanisms and eventually generate a vaccine-like response. The embodiments to this effect are described in coming paragraphs.

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

In an embodiment, the present invention provides a method of increasing plant immunity, plant defenses and tolerance to insect and fungal vectored pathogens, increasing plant disease or pest resistance, protecting plants from disease, or improving plant disease management, by modulating gene expression of plant defence genes, said method comprising contacting said plant with laminarin.

In an embodiment, the present invention provides a method of increasing plant immunity, plant defenses and tolerance to insect and fungal vectored pathogens, increasing plant disease or pest resistance, protecting plants from disease, or improving plant disease management, by modulating gene expression of plant defence genes, said method comprising applying a composition comprising laminarin to said plant. As used herein, the term “contacting” includes both direct contact (applying the compositions or active ingredient laminarin directly to the plant—typically to the foliage, stem or roots of the plant) and indirect contact (applying the compositions or active ingredient laminarin to the locus, i.e. habitat, growing ground, plant, seed, soil, area, material or environment in which the plant is growing or may grow, of the plant).

According to an embodiment, genes modulated are related to functions such as Structure of the cytoskeleton in the cell (control), Ubiquitination during plant immune signalling, NBS-LRR resistance gene (HR signaling), Gene expression regulation by stress signal transduction pathways, resistance related gene, Ethylene precursor, HR gene pathway, Regulated jasmonic acid-signals defense responses, Induced by pathogen, regulator of primed immunity, Up-regulated in viral infected grapevines, PR protein, Gene silencing as protection, Active plant defense genes (PR protein).

According to an embodiment, the plant defence genes that are modulated are selected from a-Tubulin, E3 Ubiquitin-protein ligase march3, Nbs-Irr resistance protein, AP2-like-ethylene-responsive transcription factor, Avr/Cf9 rapidly elicited protein 75, 1-aminocryclopropane-1-carboxylate oxidase-like protein, Hypersensitive response assisting protein, Nac transcription factor 29-like, Subtilisin-like protease-like, Tropinone reductase homolog at 1g07440-like, Pathogenesis-related protein P4, PR protein, AY093595.1, Regulator of gene silencing, Pti6, or variants thereof.

“Variants” is intended to mean substantially similar sequences. For polynucleotides/genes, a variant comprises a deletion and/or addition of one or more nucleotides at one or more internal sites within the native polynucleotide/gene and/or a substitution of one or more nucleotides at one or more sites in the native polynucleotide/gene. As used herein, a “native” polynucleotide or polypeptide comprises a naturally occurring nucleotide sequence or amino acid sequence, respectively. Generally, variants of a particular polynucleotide/gene of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein. Variants of a particular polynucleotide/gene of the invention (i.e., the reference polynucleotide) can also be evaluated by comparison of the percent sequence identity between the polypeptide encoded by a variant polynucleotide/gene and the polypeptide encoded by the reference polynucleotide/gene. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more internal sites in the native protein and/or substitution of one or more amino acids at one or more sites in the native protein. Variant proteins encompassed by the present invention are biologically active, that is they continue to possess the desired biological activity of the native protein, that is, regulate transcription as described herein. Such variants may result from, for example, genetic polymorphism or from human manipulation.

Plant defence genes modulated according to the invention may include the following (other than a-tubulin, which was used as a reference gene). Expression of any one, two, three, four, five, six, seven, eight, nine, ten or more of these genes may be modulated. In an embodiment, genes are overexpressed or upregulated. In an embodiment, genes are underexpressed or downregulated.

TABLE 1

| Gene | | Gene ID or | |
|---|---|---|---|
| Number | Description | accession number | Function |
| 1 | a-Tubulin | Solyc04g077020.2 | Structure of the |
| | | | cytoskeleton in the cell |
| | | | (control) |
| 2 | E3 Ubiquitin-protein | Solyc01g079530.2 | Ubiquitination during |
| | ligase march3 | | plant immune signaling |
| 3 | Nbs-Irr resistance | Solyc05g009760.1 | NBS-LRR resistance |
| | protein | | gene (HR signaling) |
| 4 | AP2-like-ethylene- | Solyc11g072600.1 | Gene expression |
| | responsive | | regulation by stress |
| | transcription factor | | signal transduction |
| | | | pathways |
| 5 | Avr/Cf9 rapidly | Solyc11g010250.1 | resistance related gene |
| | elicited protein 75 | | |
| 6 | 1-aminocryclopropane- | Solyc11g072100.1 | Ethylene precursor |
| | 1-carboxylate | | |
| | oxidase- like protein | | |
| 7 | Hypersensitive | Solyc07g006570.2 | HR gene pathway |
| | response | | |
| | assisting protein | | |
| 8 | Nac transcription | Solyc05g007770.2 | Regulated jasmonic acid- |
| | factor 29-like | | signals defense responses |
| 9 | Subtilisin-like | Solyc01g087800.2 | Induced by pathogen, |
| | protease-like | | regulator of primed |
| | | | immunity |
| 10 | Tropinone reductase | Solyc06g083470.2 | Up-regulated in viral |
| | homolog at 1g07440- | | infected grapevines |
| | like | | |
| 11 | Pathogenesis-related | M69247.1 | PR protein |
| | protein P4 | | |
| 12 | PR protein | M69248.1 | PR protein |
| 13 | AY093595.1 | AY093595.1 | PR protein |
| 14 | Regulator of gene | AY642285.1 | Gene silencing as |
| | silencing | | protection |
| 15 | Pti6 | U89257.1 | Active plant defense |
| | | | genes (PR protein) |

The polynucleotides/genes of the invention may be altered in various ways including amino acid substitutions, deletions, truncations, and insertions. Methods for such manipulations are generally known in the art. Thus, the genes and polynucleotides of the invention include both the naturally occurring sequences as well as mutant forms. Likewise, the proteins of the invention encompass both naturally occurring proteins as well as variations and modified forms thereof.

E3 Ubiquitin-protein ligase march3 is a gene involved in the ubiquitination during plant immune signalling. Ubiquitination is a proteolytic mechanism that has been demonstrated be implicated in several pathways included those mediating responses to pathogens. Many enzymes are involved in the process of ubiquitination, ubiquitin-activating (E1), conjugating (E2), and ligating (E3) enzymes. E3 ubiquitin-protein ligase may be involved in endosomal trafficking. E3 ubiquitin ligases accept ubiquitin from an E2 ubiquitin-conjugating enzyme in the form of a thioester and then directly transfer the ubiquitin to targeted substrates. The JA signaling negative regulators JAZs are the substrates of SCFCOI1 E3 ligase complexes, which transmit the JA signal to modulate plant defense responses against bacterial and biotrophic pathogens. E3 Ubiquitin-protein ligase march3 is postulated to be upregulated during virus attack.

The ACO gene family encodes 1-aminocryclopropane-1-carboxylate oxidase-like protein which is the direct precursor of the hormone ethylene. Ethylene has been shown to play an important role in the plant's defence against biotic and abiotic stress factors. The level of this molecule in the tissues is correlated to various types of stress including chilling, heat, nutrient deprivation, anaerobiosis, wounding, and pathogen infection. When plants are infected by pathogens, a large portion of the damage that occurs to the plant is due to autocatalytic ethylene synthesis and not from direct pathogen action. Also, an increasing amount of evidence has been gathered over the years, which shows that ACO is the rate-limiting step in ethylene production during certain dedicated processes.

PR5 protein are a class of proteins activated by SA pathway that stimulates the transcription of NPR1 (non-expressor of pathogen-related gene 1) which in turn leads to activation as well as accumulation SA signature gene (PR1, PR2 and PR5) products locally as well as systematically leading to systemic acquired resistance (SAR). Pathogenesis-related proteins (PR), are induced systemically and accumulated locally both at the site of infection and in distal parts of the plants, leading to development of the hypersensitive reaction (HR) and systemic acquired resistance (SAR). PR5 family is known as thaumatin-like proteins (TLPs). PR5 proteins of the high molecular weight group exhibit antifungal activities and are found in cell vacuoles. The smaller TLPs located in the extracellular are also involved in plant defense against fungal infection. PR5 genes have been identified from many plants, such as Prunus domestica, Brassica rapa, corn, poplar, and so on, and it is established that PR5 protein could obviously improve plant disease resistance. Overexpression of PR genes (chitinase, glucanase, thaumatin, defensin and thionin) individually or in combination have greatly uplifted the level of defense response in plants against a wide range of pathogens.

Hypersensitive response assisting protein is associated with hypersensitive cell death (HCD). Plants use several defense pathways to counteract pathogen invasion. The most powerful defense system of plants against invading pathogens is the hypersensitive cell death (HCD). The HCD is a highly concerted complex defense response that results in local accumulation of pathogenesis related (PR) proteins, deposition of callose and lignin in the cell wall near the infection site, and a rapid localized cell death occurs at the point of pathogen penetration. HCD, also referred as hypersensitive response (HR), is associated with disease resistance. Hypersensitive response (HR) is a form of programmed cell death (PCD) at the site of pathogen infection, which is thought to quarantine biotrophic pathogens at the site of pathogen entry and thus prevent spreading outward towards healthy tissue. Very often, the resistance mediated by R genes is due to them inducing HR, which leads to apoptosis. Most plant R genes encode NOD-like receptor (NLR) proteins. NLR protein domain architecture consists of an NB-ARC domain which is a nucleotide-binding domain, responsible for conformational changes associated with the activation of the NLR protein. In the inactive form, the NB-ARC domain is bound to Adenosine diphosphate (ADP). When a pathogen is sensed, the ADP is exchanged for Adenosine triphosphate (ATP) and this induces a conformational change in the NLR protein, which results in HR. At the N-terminus, the NLR either has a Toll-Interleukin receptor (TIR) domain (also found in mammalian toll-like receptors) or a coiled-coil (CC) motif. Both TIR and CC domains are implicated in causing cell death during HR. The C-terminus of the NLRs consists of a leucine-rich repeat (LRR) motif, which is involved in sensing the pathogen virulence factors. HR can be activated in two main ways: directly and indirectly. Direct binding of the virulence factors to the NLRs can result in the activation of HR. A ‘gene-for-gene’ relationship between the plant and pathogen that determines whether the interaction will result in disease or resistance/HR. Each dominant resistance gene (R-gene) in the host corresponded with a dominant avirulence gene (Avr gene) in the pathogen. Resistance was only conferred if both the R-gene and the corresponding Avr gene were present in the same interaction.

A related class of proteins known as pattern recognition receptors (PRRs) encode membrane-bound proteins that recognize broadly conserved microbial molecules known as microbe- or pathogen-associated molecular patterns (MAMPs or PAMPs). Upon recognition they activate a defence response known as MAMP/PAMP-triggered immunity (MTI/PTI) that is qualitatively similar to that induced by NLR, though quantitatively reduced and usually not including programmed cell death.

Subtilisin-like proteases (subtilases) are serine proteases characterized by a catalytic triad of the three amino acids, aspartate, histidine, and serine. In tomato, have been described 15 genes coding for subtilases, so far. Plant subtilases present a broad spectrum of biological functions, being involved not only in all aspects of the plant life cycle (development of seeds and fruits, cell wall modification) but also in the response to biotic and abiotic stress. Several subtilases are specifically induced following pathogen infection. In plant-pathogen interactions, the first evidence for the importance of plant subtilisin-like proteins was reported in tomato, where expression of the subtilases P69B and P69C was induced following pathogen attack and salicylic acid (SA) application. Subtilisin-like proteins are shown to exert important functions in pathogen recognition and initiation of signaling cascades leading to the activation of defense-related genes. Subtilases are especially abundant in plants, with 63 genes known in the Oryza sativa, 56 genes in Arabidopsis thaliana and at least 15 in Lycopersicon esculentum genomes.

Regulator of gene silencing protein—RNA silencing is an RNA-based gene-inactivation system that plays an important role as antiviral defense mechanism in plant and invertebrates. It is activated by the accumulation of double-stranded RNAs (dsRNAs) which are cleaved by an RNase III-like nuclease called DICER (DCL) into small-interfering RNA (siRNA). The siRNAs are 21-25 nucleotides long and they are the key mediators of RNA silencing related pathways in plants and other eukaryotic organisms. One strand of the siRNAs is incorporated in the RNA induced silencing complex (RISC) to guide the Argonaute (AGO) proteins to target RNA molecules in sequence-specific manner. The targeted RNAs are degraded or inhibited. In plants the silencing signal is amplified by the activity of host-encoded RNA-dependent RNA polymerases (RDRs) which act for a secondary siRNA production. In the model plant A. thaliana there are four different DCLs, ten AGOs and six RDRs which are specialized for different silencing-related pathways. After the discovery of RNA silencing defence mechanism it was known that RNA silencing plays a key role in the development of plant recovery from virus disease. It is generally assumed that the virus causing the initial symptoms had activated viral RNA silencing that inhibits the spread of the infection into the upper leaves and caused them to be specifically immune to secondary infection of the same virus.

Avr/Cf9 rapidly elicited protein (ACRE) genes are predicted to encode regulatory proteins, including protein kinases and transcription factors.

Transcription factor involved in the ethylene pathway-he virus invasion is correlated with higher ethylene synthesis; it is suggesting that ACO genes are involved in the response against pathogen attack. The Pti4, Pti5, and Pti6 proteins interact with the product of the Pto disease resistance gene, a Ser-Thr protein kinase. They belong to the ethylene-response factor (ERF) family of plant-unique transcription factors and bind specifically to the GCC-box cis element present in the promoters of many pathogenesis-related (PR) genes.

According to an embodiment, the effect of the modulation is immediate and starts as soon as the composition comprising laminarin is applied to the plant.

According to an embodiment, the effect of modulation may begin after a lag period.

According to another embodiment, the duration of modulatory effect of laminarin may last from a duration ranging from short-term to long-term/long-lasting. The short-term effect may range from one day to two weeks, for example up to one week or up to two weeks. The long-term effect may range from two weeks to many years, for example up to four weeks, up to three months, up to six months, up to one year, up to two years, up to five years or longer. In an embodiment, the modulatory effect of laminarin may be permanent to generate life-long defenses and tolerance to insect and fungal vectored pathogens in plants.

According to an embodiment, the present invention provides a method of controlling a viral disease or a disease caused by insect and fungal vectored pathogens in a plant, comprising applying a composition comprising laminarin to said plant, wherein laminarin modulates gene expression of plant defence genes in said plant.

In an embodiment, the present invention provides a composition comprising laminarin for increasing plant immunity, plant defenses and tolerance to insect and fungal vectored pathogens, increasing plant disease or pest resistance, protecting plants from disease, or improving plant disease management, by modulating gene expression of plant defence genes.

In an embodiment, the composition of the present invention may comprise from about 0.1% to 99% laminarin. Preferably, the composition may comprise about 0.1% to 80% laminarin.

In an embodiment, the concentration of laminarin in the said composition ranges from 10 g/L to 1000 g/L.

In an embodiment, the compositions of the present invention may additionally comprise formulation auxiliaries. Any suitable known formulation auxiliaries may be used.

The term “formulation auxiliary” or “formulation auxiliaries” or “agrochemically suitable excipient carrier” within the meaning of the invention is auxiliaries suitable for the formulation of pesticides or biosolutions, such as further solvents and/or carriers and/or surfactants (ionic or non-ionic surfactants, adjuvants, dispersing agents) and/or preservatives and/or antifoaming agents and/or anti-freezing agents.

The agrochemically suitable excipient carrier may be any one or a combination of adjuvants, co-solvents, surfactants, colorants, emulsifiers, thickeners, antifreeze agents, biocides, anti-foam agents, stabilizers, wetting agents or a mixture thereof which may be optionally added to the compositions of the present invention.

The surfactants may be selected from non-ionic, anionic or cationic surfactants.

Examples of nonionic surfactants include polyarylphenol polyethoxy ethers, polyalkylphenol polyethoxy ethers, polyglycol ether derivatives of saturated fatty acids, polyglycol ether derivatives of unsaturated fatty acids, polyglycol ether derivatives of aliphatic alcohols, polyglycol ether derivatives of cycloaliphatic alcohols, fatty acid esters of polyoxyethylene sorbitan, alkoxylated vegetable oils, alkoxylated acetylenic diols, polyalkoxylated alkylphenols, fatty acid alkoxylates, sorbitan alkoxylates, sorbitol esters, C8-C22 alkyl or alkenyl polyglycosides, polyalkoxy styrylaryl ethers, alkylamine oxides, block copolymer ethers, polyalkoxylated fatty glyceride, polyalkylene glycol ethers, linear aliphatic or aromatic polyesters, organo silicones, polyaryl phenols, sorbitol ester alkoxylates, polyalkylene oxide block copolymers, acrylic copolymers and mono- and diesters of ethylene glycol and mixtures thereof.

Examples of anionic surfactants include alcohol sulfates, alcohol ether sulfates, alkylaryl ether sulfates, alkylaryl sulfonates such as alkylbenzene sulfonates and alkylnaphthalene sulfonates and salts thereof, alkyl sulfonates, mono- or di-phosphate esters of polyalkoxylated alkyl alcohols or alkylphenols, mono- or di-sulfosuccinate esters of C12-C15 alkanols or polyalkoxylated C12-C15 alkanols, alcohol ether carboxylates, phenolic ether carboxylates, polybasic acid esters of ethoxylated polyoxyalkylene glycols consisting of oxybutylene or the residue of tetrahydrofuran, sulfoalkylamides and salts thereof such as N-methyl-N-oleoyltaurate Na salt, polyoxyalkylene alkylphenol carboxylates, polyoxyalkylene alcohol carboxylates alkyl polyglycoside/alkenyl succinic anhydride condensation products, alkyl ester sulfates, napthalene sulfonates, naphthalene formaldehyde condensates, alkyl sulfonamides, sulfonated aliphatic polyesters, sulfate esters of styrylphenyl alkoxylates, and sulfonate esters of styrylphenyl alkoxylates and their corresponding sodium, potassium, calcium, magnesium, zinc, ammonium, alkylammonium, diethanolammonium, or triethanolammonium salts, salts of ligninsulfonic acid such as the sodium, potassium, magnesium, calcium or ammonium salt, polyarylphenol polyalkoxyether sulfates and polyarylphenol polyalkoxyether phosphates, and sulfated alkyl phenol ethoxylates and phosphated alkyl phenol ethoxylates.

Cationic surfactants include alkanol amides of C8-C18 fatty acids and C8-C18 fatty amine polyalkoxylates, C10-C18 alkyldimethylbenzylammonium chlorides, coconut alkyldimethylaminoacetic acids, and phosphate esters of C8-C18 fatty amine polyalkoxylates.

Emulsifiers which can be advantageously employed herein can be readily determined by those skilled in the art and include various non-ionic, anionic, cationic and amphoteric emulsifiers, or a blend of two or more emulsifiers. Examples of nonionic emulsifiers useful in preparing the emulsifiable concentrates include the polyalkylene glycol ethers and condensation products of alkyl and aryl phenols, aliphatic alcohols, aliphatic amines or fatty acids with ethylene oxide, propylene oxides such as the ethoxylated alkyl phenols and carboxylic esters solubilized with the polyol or polyoxyalkylene. Cationic emulsifiers include quaternary ammonium compounds and fatty amine salts. Anionic emulsifiers include the oil-soluble salts (e.g., calcium) of alkylaryl sulfonic acids, oil-soluble salts or sulfated polyglycol ethers and appropriate salts of phosphated polyglycol ether.

In an embodiment, colorants may be selected from iron oxide, titanium oxide and Prussian Blue, and organic dyestuffs, such as alizarin dyestuffs, azo dyestuffs or metal phthalocyanine dyestuffs, and trace elements, such as salts of iron, manganese, boron, copper, cobalt, molybdenum and zinc.

Another embodiment involves addition of a thickener or binder which may be selected from but not limited to molasses, granulated sugar, alginates, karaya gum, jaguar gum, tragacanth gum, polysaccharide gum, mucilage, xanthan gum or combination thereof. In another embodiment, the binder may be selected from silicates such as magnesium aluminium silicate, polyvinyl acetates, polyvinyl acetate copolymers, polyvinyl alcohols, polyvinyl alcohol copolymers, celluloses, including ethylcelluloses and methylcelluloses, hydroxymethyl celluloses, hydroxypropylcelluloses, hydroxymethylpropyl-celluloses, polyvinylpyrolidones, dextrins, malto-dextrins, polysaccharides, fats, oils, proteins, gum arabics, shellacs, vinylidene chloride, vinylidene chloride copolymers, calcium lignosulfonates, acrylic copolymers, starches, polyvinylacrylates, zeins, gelatin, carboxymethylcellulose, chitosan, polyethylene oxide, acrylimide polymers and copolymers, polyhydroxyethyl acrylate, methylacrylimide monomers, alginate, ethylcellulose, polychloroprene and syrups or mixtures thereof; polymers and copolymers of vinyl acetate, methyl cellulose, vinylidene chloride, acrylic, cellulose, polyvinylpyrrolidone and polysaccharide; polymers and copolymers of vinylidene chloride and vinyl acetate-ethylene copolymers; combinations of polyvinyl alcohol and sucrose; plasticizers such as glycerol, propylene glycol, polyglycols.

In another embodiment, antifreeze agent(s) added to the composition may be alcohols selected from the group comprising of but not limited to ethylene glycol, 1,2-propylene glycol, 1,3-propylene glycol, 1,2-butanediol, 1,3-butanediol, 1,4-butanediol, 1,4-pentanediol, 3-methyl-1,5-pentanediol, 2,3-dimethyl-2,3-butanediol, trimethylol propane, mannitol, sorbitol, glycerol, pentaerythritol, 1,4-cyclohexanedimethanol, xylenol, bisphenols such as bisphenol A or the like. In addition, ether alcohols such as diethylene glycol, triethylene glycol, tetraethylene glycol, polyoxyethylene or polyoxypropylene glycols of molecular weight up to about 4000, diethylene glycol monomethylether, diethylene glycol monoethylether, triethylene glycol monomethylether, butoxyethanol, butylene glycol monobutylether, dipentaerythritol, tripentaerythritol, tetrapentaerythritol, diglycerol, triglycerol, tetraglycerol, pentaglycerol, hexaglycerol, heptaglycerol, octaglycerol.

According to an embodiment, biocides may be selected from benzothiazoles, 1,2-benzisothiazolin-3-one, sodium dichloro-s-triazinetrione, sodium benzoate, potassium sorbate, 1,2-phenyl-isothiazolin-3-one, inter chloroxylenol paraoxybenzoate butyl.

According to an embodiment, antifoam agent may be selected from Polydimethoxysiloxane, polydimethylsiloxane, Alkyl poly acrylates, Castor Oil, Fatty Acids, Fatty Acids Esters, Fatty Acids Sulfate, Fatty Alcohol, Fatty Alcohol Esters, Fatty Alcohol Sulfate, Foot Olive Oil, Mono & Di Glyceride, Paraffin Oil, Paraffin Wax, Poly Propylene Glycol, Silicones Oil, Vegetable & Animal Fats, Vegetable & Animal Fats Sulfate, Vegetable & Animal Oil, Vegetable & Animal Oil Sulfate, Vegetable & Animal Wax, Vegetable & Animal Wax Sulfate, agents based on silicon or magnesium stearate.

Representative organic liquids which can be employed in preparing the emulsifiable concentrates of the present invention are the aromatic liquids such as xylene, propyl benzene fractions, or mixed naphthalene fractions, mineral oils, substituted aromatic organic liquids such as dioctyl phthalate, kerosene, dialkyl amides of various fatty acids, particularly the dimethyl amides of fatty glycols and glycol derivatives such as the n-butyl ether, ethyl ether or methyl ether of diethylene glycol, and the methyl ether of triethylene glycol. Mixtures of two or more organic liquids are also often suitably employed in the preparation of the emulsifiable concentrate. The formulations can also contain other compatible additives, for example, plant growth regulators and other biologically active compounds used in agriculture.

The additives to be used for the formulation include, for example, a solid carrier such as kaolinite, sericite, diatomaceous earth, slaked lime, calcium carbonate, talc, white carbon, kaoline, bentonite, clay, sodium carbonate, sodium bicarbonate, mirabilite, zeolite or starch; a solvent such as water, toluene, xylene, solvent naphtha, dioxane, dimethylsulfoxide, N,N-dimethylformamide, dimethylacetamide, N-methyl-2-pyrrolidone or an alcohol; an anionic surfactant such as a salt of fatty acid, a benzoate, a polycarboxylate, a salt of alkylsulfuric acid ester, an alkyl sulfate, an alkylaryl sulfate, an alkyl diglycol ether sulfate, a salt of alcohol sulfuric acid ester, an alkyl sulfonate, an alkylaryl sulfonate, an aryl sulfonate, a lignin sulfonate, an alkyldiphenylether disulfonate, a polystyrene sulfonate, a salt of alkylphosphoric acid ester, an alkylaryl phosphate, a styrylaryl phosphate, a salt of polyoxyethylene alkyl ether sulfuric acid ester, a polyoxyethylene alkylaryl ether sulfate, a salt of polyoxyethylene alkylaryl ether sulfuric acid ester, a polyoxyethylene alkyl ether phosphate, a salt of polyoxyethylene alkylaryl phosphoric acid ester, a salt of polyoxyethylene aryl ether phosphoric acid ester, a naphthalene sulfonic acid condensed with formaldehyde or a salt of alkylnaphthalene sulfonic acid condensed with formaldehyde; a nonionic surfactant such as a sorbitan fatty acid ester, a glycerin fatty acid ester, a fatty acid polyglyceride, a fatty acid alcohol polyglycol ether, acetylene glycol, acetylene alcohol, an oxyalkylene block polymer, a polyoxyethylene alkyl ether, a polyoxyethylene alkylaryl ether, a polyoxyethylene styrylaryl ether, a polyoxyethylene glycol alkyl ether, polyethylene glycol, a polyoxyethylene fatty acid ester, a polyoxyethylene sorbitan fatty acid ester, a polyoxyethylene glycerin fatty acid ester, a polyoxyethylene hydrogenated castor oil or a polyoxypropylene fatty acid ester; and a vegetable oil or mineral oil such as olive oil, kapok oil, castor oil, palm oil, camellia oil, coconut oil, sesame oil, corn oil, rice bran oil, peanut oil, cottonseed oil, soybean oil, rapeseed oil, linseed oil, tung oil or liquid paraffins. These additives may suitably be selected for use alone or in combination as a mixture of two or more of them, so long as the object of the present invention is met. Further, additives other than the above-mentioned may be suitably selected for use among those known in this field. For example, various additives commonly used, such as a filler, a thickener, an anti-settling agent, an anti-freezing agent, a dispersion stabilizer, a safener, an anti-mold agent, a bubble agent, a disintegrator and a binder, may be used.

The agrochemical formulation may also comprise one or more antioxidants. Preferably, the agrochemical formulation comprises an antioxidants. Antioxidants are, for example, amino acids (e.g. glycine, histidine, tyrosine, tryptophan) and derivatives thereof, imidazole and imidazole derivatives (e.g. urocanic acid), peptides, such as, for example, D,L-carnosine, D-carnosine, L-carnosine and derivatives thereof (e.g. anserine), carotenoids, carotenes (e.g. α-carotene, β-carotene, lycopene) and derivatives thereof, lipoic acid and derivatives thereof (e.g. dihydrolipoic acid), aurothioglucose, propylthiouracil and further thio compounds (e.g. thioglycerol, thiosorbitol, thioglycolic acid, thioredoxin, glutathione, cysteine, cystine, cystamine and the glycosyl, N-acetyl, methyl, ethyl, propyl, amyl, butyl, lauryl, palmitoyl, oleyl, y-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol/kg to pmol/kg), also metal chelating agents (e.g. a-hydroxy fatty acids, EDTA, EGTA, phytic acid, lactoferrin), a-hydroxy acids (e.g. citric acid, lactic acid, malic acid), humic acids, bile acid, bile extracts, gallic esters (e.g. propyl, octyl and dodecyl gallate), flavonoids, catechins, bilirubin, biliverdin and derivatives thereof, unsaturated fatty acids and derivatives thereof (e.g. y-linolenic acid, linoleic acid, arachidonic acid, oleic acid), folic acid and derivatives thereof, hydroquinone and derivatives thereof (e.g. arbutin), ubiquinone and ubiquinol, and derivatives thereof, vitamin C and derivatives thereof (e.g. ascorbyl palmitate, stearate, dipalmitate, acetate, Mg ascorbyl phosphates, sodium and magnesium ascorbate, disodium ascorbyl phosphate and sulfate, potassium ascorbyl tocopheryl phosphate, chitosan ascorbate), isoascorbic acid and derivatives thereof, tocopherols and derivatives thereof (e.g. tocopheryl acetate, linoleate, oleate and succinate, tocophereth-5, tocophereth-10, tocophereth-12, tocophereth-18, tocophereth-50, tocophersolan), vitamin A and derivatives (e.g. vitamin A palmitate), the coniferyl benzoate of benzoin resin, rutin, rutinic acid and derivatives thereof, disodium rutinyl disulfate, cinnamic acid and derivatives thereof (e.g. ferulic acid, ethyl ferulate, caffeeic acid), kojic acid, chitosan glycolate and salicylate, butylhydroxytoluene, butylhydroxyanisol, nordihydroguaiacic acid, nordihydroguaiaretic acid, trihydroxybutyrophenone, uric acid and derivatives thereof, mannose and derivatives thereof, selenium and selenium derivatives (e.g. selenomethionine), stilbenes and stilbene derivatives (e.g. stilbene oxide, trans-stilbene oxide). According to the invention, suitable derivatives (salts, esters, sugars, nucleotides, nucleosides, peptides and lipids) and mixtures of these specified active ingredients or plant extracts (e.g. teatree oil, rosemary extract and rosemarinic acid) which comprise these antioxidants can be used. In general, mixtures of the aforementions antioxidants are possible.

According to an embodiment, examples of suitable solvents are water, aromatic solvents (for example Solvesso products, xylene), paraffins (for example mineral oil fractions such as kerosene or diesel oil), coal tar oils and oils of vegetable or animal origin, aliphatic, cyclic and aromatic hydrocarbons, for example toluene, xylene, paraffin, tetrahydronaphthalene, alkylated naphthalenes or their derivatives, alcohols (for example methanol, butanol, pentanol, benzyl alcohol, cyclohexanol), ketones (for example cyclohexanone, gamma-butyrolactone), pyrrolidones (NMP, NEP, NOP), acetates (glycol diacetate), glycols, fatty acid dimethylamides, fatty acids and fatty acid esters, isophorone and dimethylsulfoxide. In principle, solvent mixtures may also be used.

According to an embodiment, suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ethers, ethoxylated isooctylphenol, octylphenol, nonylphenol, alkylphenol polyglycol ethers, tributylphenyl polyglycol ethers, tristearylphenyl polyglycol ethers, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

According to an embodiment, examples of suitable carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders, polyvinylpyrrolidone and other solid carriers.).

Suitable preservatives are for example 1,2-benzisothiazolin-3-one and/or 2-Methyl-2H-isothiazol-3-one or sodium benzoate or benzoic acid.

According to an embodiment, green solvents may be used as formulation auxiliary.

As used herein the term “green solvent” refers to environmentally friendly solvents, or biosolvents. It encompasses solvents which are environmentally suitable, favourable or preferable. The green solvent may be derived from processing of biological material, agricultural material or from synthetic process. Green solvents are also referred to as green chemistry and are not necessarily limited to use with agrochemicals.

Green solvents may be selected from ethyl lactate, lactate esters, carboxylic acid amides such as lactic acid amide, carboxylic acid diamides, alkylcarboxylic acid dimethylamides such as dimethylamide of natural lactic acid (lactic acid dimethylamide).

As used herein, the term “plant” or “target plant” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, grain, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced or heterologous polynucleotides disclosed herein.

The present invention may be used for modulation of gene expression in any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa {Medicago sativa), rice {Oryza sativa), rye {Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solarium tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

A “plant” or “subject plant” or “target plant” is one in which an alteration, such as modulation of gene expression, has occurred or is intended to occur, or is a plant which is descended from a plant so altered and which comprises the alteration. A “control” or “control plant” provides a reference point for measuring changes in phenotype of the subject plant.

A control plant may comprise, for example: (a) a wild-type plant, i.e., of the same genotype as the starting material for the alteration which resulted in the subject plant; (b) a plant genetically identical to the subject plant or plant cell but which is not exposed to conditions or stimuli that would modulate expression of the gene of interest; or (c) the subject plant itself, under conditions in which the gene of interest is not modulated.

An aspect of the present invention provides a composition for modulating gene expression of plant defence genes, the said composition comprising laminarin. An embodiment provides a method of characterising gene expression of plant defence genes effected by applying a composition comprising laminarin to the said plant, comprising:
a) extraction of genetic material from the subject plant;
b) optionally, amplification of the said genetic material; and
c) analysis of the expression levels of the said genetic material;
wherein an increased level of expression of a putative plant defence gene compared to a control indicates that laminarin is eliciting overexpression or upregulation of said gene, or a decreased level of expression of a putative plant defence gene compared to a control indicates that laminarin is eliciting underexpression or downregulation of said gene.

The amplification and analysis methods may involve any known techniques, for example polymerase chain reaction (PCR), gel electrophoresis and blotting techniques such as Western Blot or Northern Blot or Southern Blot. In an embodiment, the said amplification is carried out using a set of forward and backward primers. In an embodiment, the said amplification technique is qPCR.

In an embodiment, the said method involves extraction of DNA and/or RNA from the subject plant. In an embodiment, the step a) further involves a cDNA synthesis, provided the extracted genetic material is RNA.

According to an embodiment, the present compositions can be applied to a locus by the use of conventional ground sprayers, granule applicators, watering (drenching), drip irrigation, in furrow application, spraying, atomizing, broadcasting, dusting, foaming, spreading-on, granular application, aerial methods of spraying, aerial methods of application, side dressing, spot application, ring application, root zone application, pralinage, seedling root dip, seed treatment, trunk/stem injection, padding, swabbing, root feeding, soil drenching, capsular placement, baiting, fumigation, banding, foliar application, basal application, space treatment, enclosed space fumigation, methods utilizing application using modern technologies such as, but not limited to, drones, robots, predosage device, a knapsack sprayer, a spray tank, a spray plane, or an irrigation system and by such other conventional means known to those skilled in the art.

In an embodiment, the amount of laminarin applied is in the range of 20 g ai/ha and 200 g ai/ha.

According to an embodiment, the present invention is capable of modulating plant defence genes to generate defence and tolerance against viruses/viral diseases pests such as, but not limited to, viruses from the order of Bunyavirales, Mononegavirales, Ortervirales, Picornavirales, Serpentovirales, Tymovirales; viruses from the families Alphaflexiviridae, Amalgaviridae, Aspiviridae, Benyviridae, Betaflexiviridae, Bromoviridae, Caulimoviridae, Closteroviridae, Endornaviridae, Fimoviridae, Geminiviridae, Genomoviridae, Kitaviridae, Luteoviridae, Nanoviridae putative, Phenuiviridae, Pospiviroidae, Potyviridae, Potyviridae putative, Reoviridae, Reoviridae putative, Rhabdoviridae, Secoviridae, Secoviridae putative, Solemoviridae, Tombusviridae, Tospoviridae, Totiviridae, Tymoviridae, Virgaviridae; viruses of the genera Alfamovirus, Allexivirus, Alphacarmovirus, Alphaendornavirus, Alphanecrovirus, Alphanecrovirus putative, Amalgavirus unclassified, Ampelovirus, Ampelovirus unclassif, Ampelovirus unclassified, Apscaviroid, Badnavirus, Badnavirus putative, Begomovirus, Begomovirus unclas., Benyvirus, Betacarmovirus, Brambyvirus putative, Bromovirus, Bymovirus, Capillovirus, Carlavirus, Carlavirus unclassified, Caulimovirus, Caulimovirus putative, Cavemovirus, Cilevirus, Cilevirus putative, Closterovirus, Closterovirus unclassified, Coleviroid, Comovirus, Comovirus unclassified, Crinivirus, Cucumovirus, Curtovirus putative, Cytorhabdovirus, Cytorhabdovirus unclassified, Dichorhavirus, Dichorhavirus unclassified, Emaravirus, Enamovirus putative, Fijivirus, Foveavirus, Furovirus, Gemycircularvirus, Hordeivirus, Hostuviroid, Ilarvirus, Ilarvirus unclassified, Ipomovirus, Luteovirus, Macluravirus, Maculavirus, Marafivirus, Marafivirus unclassified, Nepovirus, Nucleorhabdovirus, Nucleorhabdovirus unclassified, Ophiovirus, Orthotospovirus, Orthotospovirus putative, Petuvirus, Polerovirus, Polerovirus putative, Pospiviroid, Potexvirus, Potexvirus unclassified, Potyvirus, Sobemovirus, Tenuivirus putative, Tobamovirus, Tobamovirus putative, Tobravirus, Totivirus putative, Trichovirus, Tritimovirus, Tymovirus, Tymovirus unclassified, Umbravirus putative, Varicosavirus, Vitivirus, Waikavirus.

Some examples of orthotospoviruses against which the invention may confer protection are as follows: Alstroemeria necrotic streak orthotospovirus, Alstroemeria yellow spot orthotospovirus, Bean necrotic mosaic orthotospovirus, Calla lily chlorotic spot orthotospovirus, Capsicum chlorosis orthotospovirus, Chrysanthemum stem necrosis orthotospovirus, Groundnut bud necrosis orthotospovirus, Groundnut chlorotic fan spot orthotospovirus, Groundnut ringspot orthotospovirus, Groundnut yellow spot orthotospovirus, Hippeastrum chlorotic ringspot orthotospovirus, Impatiens necrotic spot orthotospovirus, Iris yellow spot orthotospovirus, Melon severe mosaic orthotospovirus, Melon yellow spot orthotospovirus, Mulberry vein banding associated orthotospovirus, Pepper chlorotic spot orthotospovirus, Polygonum ringspot orthotospovirus, Soybean vein necrosis orthotospovirus, Tomato chlorotic spot orthotospovirus, Tomato spotted wilt orthotospovirus, Tomato yellow ring orthotospovirus, Tomato zonate spot orthotospovirus, Watermelon bud necrosis orthotospovirus, Watermelon silver mottle orthotospovirus and Zucchini lethal chlorosis orthotospovirus.

Some examples of Potyvirus against which the present invention may be found effective are African eggplant mosaic virus, Algerian watermelon mosaic virus, Alstroemeria mosaic virus, Alternanthera mild mosaic virus, Amaranthus leaf mottle virus, Amazon lily mosaic virus, Angelica virus Y, Apium virus Y, Araujia mosaic virus, Arracacha mottle virus, Asparagus virus 1, Banana bract mosaic virus, Barbacena virus Y, Basella rugose mosaic virus, Bean common mosaic necrosis virus, Bean common mosaic virus, Bean yellow mosaic virus, Beet mosaic virus, Bidens mosaic virus, Bidens mottle virus, Blue squill virus A, Bramble yellow mosaic virus, Brugmansia mosaic virus, Brugmansia suaveolens mottle virus, Butterfly flower mosaic virus, Calanthe mild mosaic virus, Calla lily latent virus, Callistephus mottle virus, Canna yellow streak virus, Carnation vein mottle virus, Carrot thin leaf virus, Carrot virus Y, Catharanthus mosaic virus, Celery mosaic virus, Ceratobium mosaic virus, Chilli ringspot virus, Chilli veinal mottle virus, Chinese artichoke mosaic virus, Clitoria virus Y, Clover yellow vein virus, Cocksfoot streak virus, Colombian datura virus, Commelina mosaic virus, Cowpea aphid-borne mosaic virus, Cucurbit vein banding virus, Cypripedium virus Y, Cyrtanthus elatus virus A, Daphne mosaic virus, Daphne virus Y, Dasheen mosaic virus, Datura shoestring virus, Dendrobium chlorotic mosaic virus, Dioscorea mosaic virus, Diuris virus Y, Donkey orchid virus A, East Asian Passiflora distortion virus, East Asian Passiflora virus, Endive necrotic mosaic virus, Euphorbia ringspot virus, Freesia mosaic virus, Fritillary virus Y, Gloriosa stripe mosaic virus, Gomphocarpus mosaic virus, Habenaria mosaic virus, Hardenbergia mosaic virus, Henbane mosaic virus, Hibbertia virus Y, Hippeastrum mosaic virus, Hyacinth mosaic virus, Impatiens flower break virus, Iris fulva mosaic virus, Iris mild mosaic virus, Iris severe mosaic virus, Japanese yam mosaic virus, Jasmine virus T, Johnsongrass mosaic virus, Kalanchoe mosaic virus, Keunjorong mosaic virus, Konjac mosaic virus, Leek yellow stripe virus, Lettuce Italian necrotic virus, Lettuce mosaic virus, Lily mottle virus, Lily virus Y, Lupinus mosaic virus, Lycoris mild mottle virus, Maize dwarf mosaic virus, Malva vein clearing virus, Mashua virus Y, Meadow saffron breaking virus, Mediterranean ruda virus, Moroccan watermelon mosaic virus, Narcissus degeneration virus, Narcissus late season yellows virus, Narcissus yellow stripe virus, Nerine yellow stripe virus, Nothoscordum mosaic virus, Onion yellow dwarf virus, Ornithogalum mosaic virus, Ornithogalum virus 2, Ornithogalum virus 3, Panax virus Y, Papaya leaf distortion mosaic virus, Papaya ringspot virus, Paris mosaic necrosis virus, Parsnip mosaic virus, Passiflora chlorosis virus, Passion fruit woodiness virus, Pea seed-borne mosaic virus, Peanut mottle virus, Pecan mosaic-associated virus, Pennisetum mosaic virus, Pepper mottle virus, Pepper severe mosaic virus, Pepper veinal mottle virus, Pepper yellow mosaic virus, Peru tomato mosaic virus, Pfaffia mosaic virus, Platycodon mild mottle virus, Pleione virus Y, Plum pox virus, Pokeweed mosaic virus, Potato virus A, Potato virus V, Potato virus Y, Potato yellow blotch virus, Ranunculus leaf distortion virus, Ranunculus mild mosaic virus, Ranunculus mosaic virus, Rhopalanthe virus Y, Saffron latent virus, Sarcochilus virus Y, Scallion mosaic virus, Shallot yellow stripe virus, Sorghum mosaic virus, Soybean mosaic virus, Spiranthes mosaic virus 3, Sudan watermelon mosaic virus, Sugarcane mosaic virus, Sunflower chlorotic mottle virus, Sunflower mild mosaic virus, Sunflower mosaic virus, Sunflower ring blotch virus, Sweet potato feathery mottle virus, Sweet potato latent virus, Sweet potato mild speckling virus, Sweet potato virus 2, Sweet potato virus C, Sweet potato virus G, Tamarillo leaf malformation virus, Telfairia mosaic virus, Telosma mosaic virus, Thunberg fritillary mosaic virus, Tobacco etch virus, Tobacco mosqueado virus, Tobacco vein banding mosaic virus, Tobacco vein mottling virus, Tomato necrotic stunt virus, Tradescantia mild mosaic virus, Tuberose mild mosaic virus, Tuberose mild mottle virus, Tulip breaking virus, Tulip mosaic virus, Turnip mosaic virus, Twisted-stalk chlorotic streak virus, Vallota mosaic virus, Vanilla distortion mosaic virus, Verbena virus Y, Watermelon leaf mottle virus, Watermelon mosaic virus, Wild melon banding virus, Wild onion symptomless virus, Wild potato mosaic virus, Wild tomato mosaic virus, Wisteria vein mosaic virus, Yam mild mosaic virus, Yam mosaic virus, Yambean mosaic virus, Zantedeschia mild mosaic virus, Zea mosaic virus, Zucchini shoestring virus, Zucchini tigre mosaic virus, Zucchini yellow fleck virus and Zucchini yellow mosaic virus.

Some examples of Tobamovirus against which the present invention may exert efficacy are Bell pepper mosaic virus (BPeMV), Brugmansia mild mottle virus, Cactus mild mottle virus (CMMoV), Clitoria yellow mottle virus, Cucumber fruit mottle mosaic virus, Cucumber green mottle mosaic virus (CGMMV), Cucumber mottle virus, Frangipani mosaic virus (FrMV), Hibiscus latent Fort Pierce virus (HLFPV), Hibiscus latent Singapore virus (HLSV), Kyuri green mottle mosaic virus, Maracuja mosaic virus (MarMV), Obuda pepper virus (ObPV), Odontoglossum ringspot virus (ORSV), Opuntia chlorotic ringspot virus, Paprika mild mottle virus, Passion fruit mosaic virus, Pepper mild mottle virus (PMMoV), Plumeria mosaic virus, Rattail cactus necrosis-associated virus (RCNaV), Rehmannia mosaic virus, Ribgrass mosaic virus (HRV), Sammons's Opuntia virus (SOV), Streptocarpus flower break virus, Sunn-hemp mosaic virus (SHMV), Tobacco latent virus, Tobacco mild green mosaic virus, Tomato brown rugose fruit virus (ToBRFV), Tomato mosaic virus (ToMV), Tobacco mosaic virus (T2MV)—type Species, Tomato mottle mosaic virus, Tropical soda apple mosaic virus, Turnip vein-clearing virus (TVCV), Ullucus mild mottle virus, Wasabi mottle virus (WMoV), Yellow tailflower mild mottle virus, Youcai mosaic virus (YoMV) aka oilseed rape mosaic virus (ORMV) and Zucchini green mottle mosaic virus.

In a preferred embodiment, the present invention is capable of modulating plant defence genes to generate defence and tolerance against viruses/viral diseases pests such as a virus of the genus Orthotospovirus, a virus of the genus Cucumovirus, a virus of the genus Potyvirus, a virus of the genus Caulimovirus, and a virus of the genus Tobamovirus.

In a yet another preferred embodiment, the present invention is capable of modulating plant defence genes to generate defence and tolerance against viruses/viral diseases pests such as Tomato Spotted Wilt Virus (TSWV), Cucumber Mosaic Virus (CMV), Potato Virus Y (PVY), Cauliflower Mosaic Virus (CaMV) or Tobacco Mosaic Virus (TMV).

According to an embodiment, the present invention is capable of modulating plant defence genes to generate defence and tolerance against bacteria/bacterial diseases pests such as, but not limited to, Erwinia, Pectobacterium, Pantoea, Agrobacterium, Pseudomonas, Ralstonia, Burkholderia, Acidovorax, Xanthomonas, Clavibacter, Streptomyces, Xylella, Spiroplasma, and Phytoplasma, galls and overgrowths, wilts, leaf spots, specks and blights, soft rots, scabs and cankers.

According to an embodiment, concentrated formulations can be dispersed in water, or another liquid, for application, or formulations can be dust-like or granular. The formulations are prepared according to procedures which are conventional in the agricultural chemical art, but which are novel and important because of the presence therein of a composition. The formulations that are applied most often are aqueous suspensions or emulsions. Either such water-soluble, water-suspendable, or emulsifiable formulations are solids, usually known as wettable powders, or liquids, usually known as emulsifiable concentrates, aqueous suspensions, or suspension concentrates. The present disclosure contemplates all vehicles by which the compositions can be formulated for delivery and use as an herbicide.

The compositions of the present invention may also be formulated as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ULV) liquid, ultra low volume (ULV) suspension, water dispersible granules or tablets, water dispersible powder for slurry treatment, water soluble granules or tablets, water soluble powder for seed treatment and wettable powder. More specifically, the compositions are formulated as solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF).

The compounds and mixtures according to the invention are suitable for use in seed treatment. Solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

In an embodiment, the composition of the present invention is applied directly and/or indirectly to the plant and/or to plant propagation material by drenching the soil, by drip application onto the soil, by soil injection, by dipping or by treatment of seeds.

The present invention may further be combined with herbicides, insecticides, fungicides, fertilizers, micronutrients, superabsorbent polymers or biological entities. The present invention may further be combined with synergists. The present invention comprising the additional active agents may be applied as a premix or tank-mix, simultaneously, in-parallel or in a sequential manner. Such combinations may provide a synergistic effect in modulating the expressions of plant defence genes leading to a further enhancement of immune response of the subject plants.

According to an embodiment, the present invention provides a plant or a plant propagation material with an enhanced immune response, wherein the said plant or plant propagation material has been treated with a composition comprising laminarin.

According to an embodiment, the expression of plant defence genes of the said plant or plant propagation material is modulated.

According to an embodiment, the said plant or plant propagation material is resistant to attack by pests or diseases.

In an embodiment, the said disease may be a viral disease.

The present invention is illustrated with examples. However, present invention and the above embodiments should not be construed as to be limiting to the given example.

### Example 1

Plant Material and Virus Inoculation

Tomato ecotype “corbarino” reported to be susceptible to TSWV infection was used in the experiment. Plants were grown in pots in a climatic chamber with a photoperiod of 16/8 hour at 25° C.

The viral strain (TSWV PV PV-0393) was utilized in the experiment. The virus was mechanically inoculated and propagated on tomato “corbarino”. The inoculum was prepared in an ice-cooled mortar grounding 1 gr of infected tomato tissue in 10 ml of Na-phosphate buffer 0.01M. The fresh prepared inoculum was stored in ice before use. Tomato plants at BBCH16 were dusted with Carborundum 600 mesh and selected leaves were rubbed with some droplets of inoculum. After 5 min. plants were sprayed with sterile water to remove the excess of inoculum and preserve the cell turgor.

Four treatments were compared: i) Mock inoculated; ii) TSWV; iii) 0.25% Laminarin; iv) 0.25% Laminarin+TSWV. In the last treatment the virus was inoculated 24 hrs after the laminarin application.

Symptoms Observation

Inoculated plants were monitored daily and to assess the viral replication the ELISA tests were carried out with a commercial TSWV DAS-ELISA diagnostic kit (Loewe Biochemica GmbH, Germany) according to manufacturer's instructions.

RNA Extraction and qRT PCR

Total RNA was extracted from systemic infected tomato leaves by using the Quick-RNA Plant Kit (Zymo Research) following the manufacturer instructions. The extractions were carried out using three plants after 12 h, 24 h, 48 h, 72 h, 7 days after inoculation. Leaves were instantly frozen in liquid nitrogen after cutting to avoid any deterioration of the sample as well as any defense reaction. The RNA concentration was quantified by measuring the absorbance at 260 nm using a Nanodrop 2000 (Thermofisher). The purity of all of the RNA samples was assessed at an absorbance ratio of OD260/280 and OD260/230, while its structural integrity was checked by agarose gel electrophoresis. Only high-quality RNA with OD 260/280 and OD 260/230>2 was used for subsequent steps. Single-stranded cDNA was synthesized from 1 μg of total RNA using an iScript™ Select cDNA Synthesis Kit and random hexamers as primers (Bio-Rad, Milan, Italy), according to the manufacturer's instructions. Three biological replicates were created from the 3 leaves collected to test 15 genes. Alpha tubulin was used as reference gene.

qPCR reactions were performed with BioRad (Applied Biosystems, Foster City, CA, USA) using SYBR R Green. Each reaction was performed in 20 μl containing 10 μl of 2× Power SYBR Green PCR Master Mix (Life Technologies), 0.2-0.3 μM primers and cDNA samples diluted 1:20. Each qPCR reaction was performed in triplicate. Reactions were run using the cycling parameter described previously (Reid et al., 2006) and the qPCR data were analyzed by the Ct method (Pfaffl, 2001). In order to analyze the qPCR data, a-tubulin gene was used as housekeeping to determine the relative expression level of the other genes analyzed in this work. For the determination of qPCR efficiency of each primer pairs, a standard curve was performed using the following cDNA dilutions: 1:4, 1:16, 1:64; 1:256 and 1:1024. Every measurement was made in triplicate. The corresponding qPCR efficiencies (E) were calculated for every primer pair with the software 7300 System SDS software (Applied Biosystems, Foster City, CA, USA) according to the equation E=(10−1/slope−1)×100 (Rutledge and Stewart, 2008).

Statistical Analysis

The significance of the differences between the gene expressions levels were compared by one-way ANOVA (p=0.01) and the means were separated by Tukey post-hoc test using SPSS ver.26 (SAS Institute Inc., 2004, Cary, NC, USA).

TABLE 2

| Gene | | Primers |
|---|---|---|
| Number | Description | (SEQ ID NOs) |
| 1 | a-Tubulin | 1/2 |
| 2 | E3 Ubiquitin-protein ligase march3 | 3/4 |
| 3 | Nbs-Irr resistance protein | 5/6 |
| 4 | AP2-like-ethylene-responsive | 7/8 |
| | transcription factor | |
| 5 | Avr/Cf9 rapidly elicited protein 75 | 9/10 |
| 6 | 1-aminocryclopropane-1-carboxylate | 11/12 |
| | oxidase-like protein | |
| 7 | Hypersensitive response assisting | 13/14 |
| | protein | |
| 8 | Nac transcription factor 29-like | 15/16 |
| 9 | Subtilisin-like protease-like | 17/18 |
| 10 | Tropinone reductase homolog at | 19/20 |
| | 1g07440-like | |
| 11 | Pathogenesis-related protein P4 | 21/22 |
| 12 | PR protein | 23/24 |
| 13 | AY093595.1 | 25/26 |
| 14 | Regulator of gene silencing | 27/28 |
| 15 | Pti6 | 29/30 |

Observations

Globally, the virus did not induce visible alterations in the first days after inoculation. In fact, TSWV inoculations induced the symptoms appearance after 11 days post inoculation (dpi) in all groups of plants. Generally, TSWV induces local symptoms on test plant (Petunia hybrida, Datura metel) in 2-4 days. No local symptoms were observed while systemic ones consisted in leaf bronzing a mild stunting; in very few cases tip necrosis was noted. The inoculation procedure was repeated twice to consolidate the results. The detection of the virus by ELISA test resulted positive in all inoculated plants after 7 days indicating a slow progression of the infection.

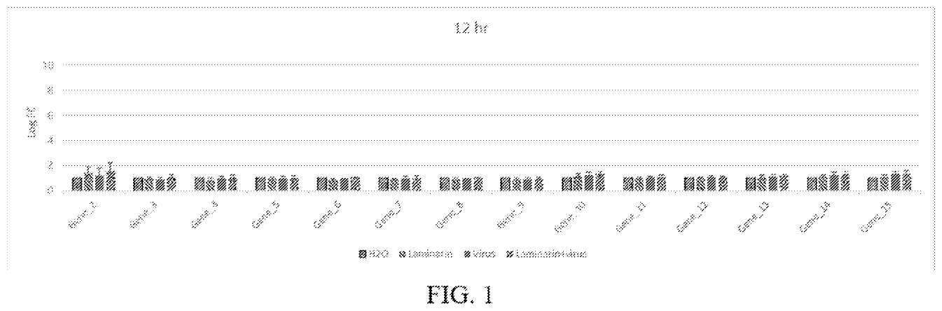

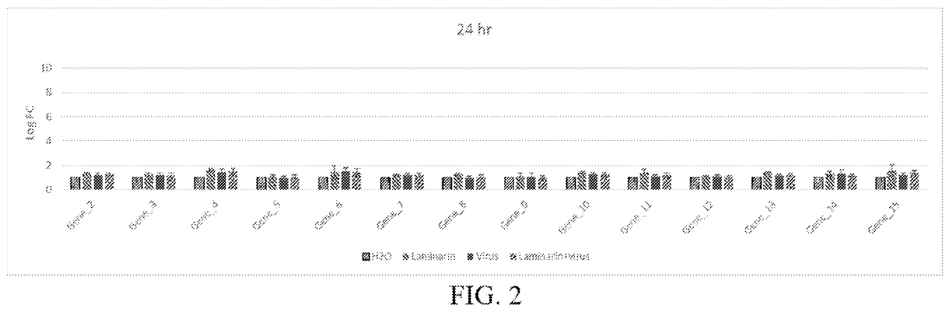

12 and 24 hr: FIG. 1 shows gene expression compared to water treatment after 12 hr from application. FIG. 2 shows gene expression compared to water treatment after 24 hr from application

Gene-expression data (FIGS. 1 and 2) indicate that 12 and 24 hrs. after the inoculation there are few statistically significant over expressions in some tested genes (2, 6 and 13). For genes 2 and 13 it is noted that the expression increases from the first to the second time point, while for gene 6 there is a decrease. After 24 hours the recorded expression of the selected genes is almost flat and the slight overexpression of those commented could also be due to the mechanical rubbing of the carborundum, which the plant perceives as a stimulus to which it reacts.

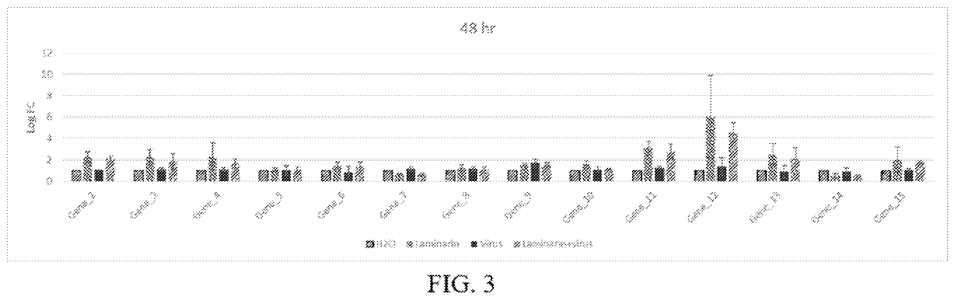

48 hr: FIG. 3 shows gene expression compared to water treatment after 48 hr from application.

After 48 hours from the treatment the increment of gene expression was confirmed for gene 2 and gene 13, even though in the second gene there is no significant different with the control. Globally, this time point indicates that the laminarin alone and in combination with the virus activates the set of defense genes at higher level compared to TSWV. Only for the gene 7, gene 9 and gene 14 this scenario is inverted.

In the first case the treatments 2 and 4 induce the downregulation while in the second case the treatment 3 induce the upregulation.

Gene 7 seems to be activated by virus infection at higher level compared to other treatments. Gene 9 is upregulated on tomato plants inoculated with TSWV.

While not being significant, the gene 12 was highly overexpressed since 48 hr post treatment. The logfold change is relevant even though the standard deviation of the data does not make the data significant. This gene code for a PR1 protein: increased expression of PR1 and PR2 genes have routinely been used as a molecular marker of SAR. The overexpression of gene 12 is slightly reduced at 72 hr but at this time point there is a statistical significance that support the idea that laminarin activate the SAR in tomato.

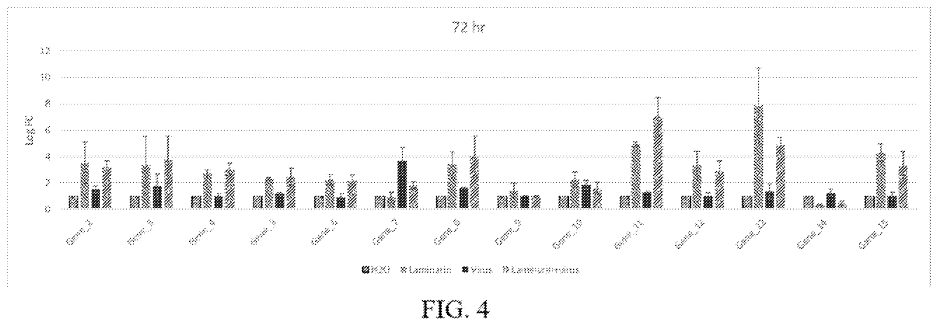

72 hr: FIG. 4 shows gene expression compared to water treatment after 72 hr from application

At 72 hr the gene expression of the tested genes gives a clear evidence of the laminarin effects. For some genes (gene 9 and gene 14) there is no clear overexpression suggesting no role after the treatment with laminarin. The gene 7 is the only tested gene where the overexpression induced by the TSWV is higher compared to the other treatments. In the gene 14 the scenario is similar but due to the down regulation induced by laminarin alone and with TSWV.

TABLE 3
Statistical analyses of RQ values after application - Means
with the same letter are not statistical different (p = 0.01)

| | | | | | | | Std | Std | |
|---|---|---|---|---|---|---|---|---|---|
| Gene | time | Treatment | RQ1 | RQ2 | RQ3 | mean | Dev | Err | |
| Gene_2 | 12 hr | H2O | 10.000 | 10.000 | 10.000 | 10.000 | 0.0000 | 0.0000 | a |
| Gene_2 | 12 hr | Laminarin | 13.140 | 19.586 | 10.706 | 14.477 | 0.4588 | 0.2649 | a |
| Gene_2 | 12 hr | Virus | 0.8845 | 19.055 | 0.7639 | 11.847 | 0.6272 | 0.3621 | a |
| Gene_2 | 12 hr | Laminarin + virus | 13.056 | 23.403 | 12.079 | 16.179 | 0.6275 | 0.3623 | a |
| Gene_2 | 24 hr | H2O | 10.000 | 10.000 | 10.000 | 10.000 | 0.0000 | 0.0000 | a |
| Gene_2 | 24 hr | Laminarin | 13.152 | 13.329 | 14.033 | 13.505 | 0.0466 | 0.0269 | b |
| Gene_2 | 24 hr | Virus | 11.490 | 13.658 | 11.208 | 12.119 | 0.1340 | 0.0774 | ab |
| Gene_2 | 24 hr | Laminarin + virus | 11.700 | 13.596 | 12.341 | 12.546 | 0.0964 | 0.0557 | ab |
| Gene_2 | 48 hr | H2O | 10.000 | 10.000 | 10.000 | 10.000 | 0.0000 | 0.0000 | a |
| Gene_2 | 48 hr | Laminarin | 27.467 | 17.321 | 23.298 | 22.695 | 0.5100 | 0.2944 | a |
| Gene_2 | 48 hr | Virus | 10.798 | 10.746 | 10.896 | 10.813 | 0.0076 | 0.0044 | b |
| Gene_2 | 48 hr | Laminarin + virus | 24.083 | 18.519 | 21.352 | 21.318 | 0.2782 | 0.1606 | b |
| Gene_2 | 72 hr | H2O | 10.000 | 10.000 | 10.000 | 10.000 | 0.0000 | 0.0000 | a |
| Gene_2 | 72 hr | Laminarin | 29.447 | 23.000 | 53.007 | 35.151 | 15.796 | 0.9120 | a |
| Gene_2 | 72 hr | Virus | 13.252 | 14.215 | 17.832 | 15.100 | 0.2415 | 0.1394 | a |
| Gene_2 | 72 hr | Laminarin + virus | 28.726 | 29.558 | 37.336 | 31.873 | 0.4749 | 0.2742 | a |
| Gene_3 | 12 hr | H2O | 10.000 | 10.000 | 10.000 | 10.000 | 0.0000 | 0.0000 | a |
| Gene_3 | 12 hr | Laminarin | 10.545 | 10.498 | 0.8329 | 0.9791 | 0.1266 | 0.0731 | a |
| Gene_3 | 12 hr | Virus | 0.9177 | 0.9376 | 0.8844 | 0.9132 | 0.0269 | 0.0155 | a |
| Gene_3 | 12 hr | Laminarin + virus | 11.935 | 12.286 | 0.9487 | 11.236 | 0.1525 | 0.0881 | a |
| Gene_3 | 24 hr | H2O | 10.000 | 10.000 | 10.000 | 10.000 | 0.0000 | 0.0000 | a |
| Gene_3 | 24 hr | Laminarin | 12.229 | 13.976 | 11.514 | 12.573 | 0.1266 | 0.0731 | a |
| Gene_3 | 24 hr | Virus | 0.9948 | 13.867 | 12.086 | 11.967 | 0.1962 | 0.1133 | a |
| Gene_3 | 24 hr | Laminarin + virus | 11.167 | 10.509 | 13.936 | 11.871 | 0.1819 | 0.1050 | a |
| Gene_3 | 48 hr | H2O | 10.000 | 10.000 | 10.000 | 10.000 | 0.0000 | 0.0000 | a |
| Gene_3 | 48 hr | Laminarin | 27.419 | 26.255 | 14.098 | 22.591 | 0.7378 | 0.4260 | a |
| Gene_3 | 48 hr | Virus | 10.071 | 12.016 | 11.722 | 11.269 | 0.1049 | 0.0605 | a |
| Gene_3 | 48 hr | Laminarin + virus | 12.976 | 26.960 | 15.208 | 18.381 | 0.7513 | 0.4337 | a |
| Gene_3 | 72 hr | H2O | 10.000 | 10.000 | 10.000 | 10.000 | 0.0000 | 0.0000 | a |
| Gene_3 | 72 hr | Laminarin | 19.725 | 58.615 | 21.699 | 33.346 | 21.905 | 12.647 | a |
| Gene_3 | 72 hr | Virus | 11.730 | 28.428 | 12.778 | 17.645 | 0.9353 | 0.5400 | a |
| Gene_3 | 72 hr | Laminarin + virus | 47.747 | 48.776 | 16.860 | 37.794 | 18.137 | 10.471 | a |
| Gene_4 | 12 hr | H2O | 10.000 | 10.000 | 10.000 | 10.000 | 0.0000 | 0.0000 | a |
| Gene_4 | 12 hr | Laminarin | 0.9931 | 0.9090 | 0.7654 | 0.8892 | 0.1151 | 0.0665 | a |
| Gene_4 | 12 hr | Virus | 10.442 | 0.9271 | 0.9725 | 0.9813 | 0.0591 | 0.0341 | a |
| Gene_4 | 12 hr | Laminarin + virus | 12.523 | 0.9104 | 10.299 | 10.642 | 0.1735 | 0.1002 | a |
| Gene_4 | 24 hr | H2O | 1.000 | 1.000 | 1.000 | 1.000 | 0.000 | 0.000 | a |
| Gene_4 | 24 hr | Laminarin | 1.744 | 1.728 | 1.516 | 1.662 | 0.127 | 0.073 | a |
| Gene_4 | 24 hr | Virus | 1.227 | 1.409 | 1.713 | 1.449 | 0.246 | 0.142 | a |
| Gene_4 | 24 hr | Laminarin + virus | 1.250 | 1.754 | 1.545 | 1.516 | 0.253 | 0.146 | a |
| Gene_4 | 48 hr | H2O | 10.000 | 10.000 | 10.000 | 10.000 | 0.0000 | 0.0000 | a |
| Gene_4 | 48 hr | Laminarin | 15.231 | 15.784 | 37.709 | 22.908 | 12.821 | 0.7402 | a |
| Gene_4 | 48 hr | Virus | 12.070 | 0.9813 | 12.357 | 11.413 | 0.1393 | 0.0804 | a |
| Gene_4 | 48 hr | Laminarin + virus | 13.802 | 15.313 | 21.904 | 17.006 | 0.4308 | 0.2487 | a |
| Gene_4 | 72 hr | H2O | 10.000 | 10.000 | 10.000 | 10.000 | 0.0000 | 0.0000 | a |
| Gene_4 | 72 hr | Laminarin | 25.807 | 30.148 | 26.311 | 27.422 | 0.2374 | 0.1371 | b |
| Gene_4 | 72 hr | Virus | 11.243 | 0.7849 | 10.893 | 0.9995 | 0.1867 | 0.1078 | a |
| Gene_4 | 72 hr | Laminarin + virus | 35.424 | 26.180 | 29.297 | 30.301 | 0.4703 | 0.2715 | b |
| Gene_5 | 12 hr | H2O | 10.000 | 10.000 | 10.000 | 10.000 | 0.0000 | 0.0000 | a |
| Gene_5 | 12 hr | Laminarin | 0.7901 | 11.285 | 0.9408 | 0.9531 | 0.1695 | 0.0979 | a |
| Gene_5 | 12 hr | Virus | 10.680 | 0.9574 | 0.8660 | 0.9638 | 0.1012 | 0.0584 | a |
| Gene_5 | 12 hr | Laminarin + virus | 0.8046 | 10.612 | 11.537 | 10.065 | 0.1809 | 0.1044 | a |
| Gene_5 | 24 hr | H2O | 10.000 | 10.000 | 10.000 | 10.000 | 0.0000 | 0.0000 | a |
| Gene_5 | 24 hr | Laminarin | 11.135 | 12.189 | 0.9994 | 11.106 | 0.1098 | 0.0634 | a |
| Gene_5 | 24 hr | Virus | 11.483 | 0.9251 | 0.8633 | 0.9789 | 0.1499 | 0.0866 | a |
| Gene_5 | 24 hr | Laminarin + virus | 10.601 | 12.023 | 0.9700 | 10.775 | 0.1171 | 0.0676 | a |
| Gene_5 | 48 hr | H2O | 10.000 | 10.000 | 10.000 | 10.000 | 0.0000 | 0.0000 | a |
| Gene_5 | 48 hr | Laminarin | 0.9204 | 12.321 | 10.531 | 10.685 | 0.1564 | 0.0903 | a |
| Gene_5 | 48 hr | Virus | 0.6993 | 15.316 | 0.7892 | 10.067 | 0.4568 | 0.2637 | a |
| Gene_5 | 48 hr | Laminarin + virus | 0.8610 | 13.586 | 10.405 | 10.867 | 0.2520 | 0.1455 | a |
| Gene_5 | 72 hr | H2O | 10.000 | 10.000 | 10.000 | 10.000 | 0.0000 | 0.0000 | a |
| Gene_5 | 72 hr | Laminarin | 22.196 | 23.867 | 23.473 | 23.179 | 0.0874 | 0.0504 | bc |
| Gene_5 | 72 hr | Virus | 12.711 | 11.102 | 10.919 | 11.577 | 0.0986 | 0.0569 | ab |
| Gene_5 | 72 hr | Laminarin + virus | 17.401 | 30.624 | 25.095 | 24.373 | 0.6641 | 0.3834 | c |
| Gene_6 | 12 hr | H2O | 1.00.000 | 1.00.000 | 1.00.000 | 1.00.000 | 0.00000 | 0.00000 | ab |
| Gene_6 | 12 hr | Laminarin | 0.90876 | 0.97097 | 0.89242 | 0.92405 | 0.04145 | 0.02393 | a |
| Gene_6 | 12 hr | Virus | 0.96004 | 0.99130 | 0.92185 | 0.95773 | 0.03479 | 0.02008 | a |
| Gene_6 | 12 hr | Laminarin + virus | 1.04.909 | 1.10.188 | 1.08.122 | 1.07.740 | 0.02660 | 0.01536 | b |
| Gene_6 | 24 hr | H2O | 10.000 | 10.000 | 10.000 | 10.000 | 0.0000 | 0.0000 | a |
| Gene_6 | 24 hr | Laminarin | 16.709 | 18.603 | 0.8182 | 14.498 | 0.5551 | 0.3205 | a |
| Gene_6 | 24 hr | Virus | 11.578 | 17.583 | 15.780 | 14.981 | 0.3081 | 0.1779 | a |
| Gene_6 | 24 hr | Laminarin + virus | 13.422 | 17.528 | 12.610 | 14.520 | 0.2636 | 0.1522 | a |
| Gene_6 | 48 hr | H2O | 10.000 | 10.000 | 10.000 | 10.000 | 0.0000 | 0.0000 | a |
| Gene_6 | 48 hr | Laminarin | 16.033 | 16.927 | 10.675 | 14.545 | 0.3381 | 0.1952 | a |
| Gene_6 | 48 hr | Virus | 0.3212 | 14.698 | 0.7899 | 0.8603 | 0.5776 | 0.3335 | a |
| Gene_6 | 48 hr | Laminarin + virus | 14.823 | 17.395 | 0.9742 | 13.987 | 0.3895 | 0.2249 | a |
| Gene_6 | 72 hr | H2O | 10.000 | 10.000 | 10.000 | 10.000 | 0.0000 | 0.0000 | a |
| Gene_6 | 72 hr | Laminarin | 27.013 | 20.615 | 19.604 | 22.410 | 0.4017 | 0.2320 | b |
| Gene_6 | 72 hr | Virus | 0.6374 | 10.823 | 10.134 | 0.9110 | 0.2395 | 0.1383 | a |
| Gene_6 | 72 hr | Laminarin + virus | 26.404 | 20.406 | 19.154 | 21.988 | 0.3875 | 0.2237 | b |
| Gene_7 | 12 hr | H2O | 10.000 | 10.000 | 10.000 | 10.000 | 0.0000 | 0.0000 | a |
| Gene_7 | 12 hr | Laminarin | 0.9749 | 0.8446 | 0.8747 | 0.8981 | 0.0682 | 0.0394 | a |
| Gene_7 | 12 hr | Virus | 0.9824 | 0.9367 | 0.9436 | 0.9543 | 0.0246 | 0.0142 | a |
| Gene_7 | 12 hr | Laminarin + virus | 12.106 | 0.8295 | 10.421 | 10.274 | 0.1910 | 0.1103 | a |
| Gene_7 | 24 hr | H2O | 10.000 | 10.000 | 10.000 | 10.000 | 0.0000 | 0.0000 | a |
| Gene_7 | 24 hr | Laminarin | 11.623 | 12.446 | 12.085 | 12.051 | 0.0413 | 0.0238 | a |
| Gene_7 | 24 hr | Virus | 12.947 | 11.161 | 12.800 | 12.303 | 0.0991 | 0.0572 | a |
| Gene_7 | 24 hr | Laminarin + virus | 13.380 | 12.897 | 10.449 | 12.242 | 0.1571 | 0.0907 | a |
| Gene_7 | 48 hr | H2O | 10.000 | 10.000 | 10.000 | 10.000 | 0.0000 | 0.0000 | ab |
| Gene_7 | 48 hr | Laminarin | 0.7103 | 0.7130 | 0.6351 | 0.6861 | 0.0442 | 0.0255 | a |
| Gene_7 | 48 hr | Virus | 11.549 | 10.682 | 13.528 | 11.920 | 0.1459 | 0.0842 | b |
| Gene_7 | 48 hr | Laminarin + virus | 0.7159 | 0.5334 | 0.8167 | 0.6886 | 0.1436 | 0.0829 | a |
| Gene_7 | 72 hr | H2O | 10.000 | 10.000 | 10.000 | 10.000 | 0.0000 | 0.0000 | a |
| Gene_7 | 72 hr | Laminarin | 10.549 | 0.4677 | 11.927 | 0.9051 | 0.3850 | 0.2223 | a |
| Gene_7 | 72 hr | Virus | 38.584 | 45.920 | 25.733 | 36.746 | 10.218 | 0.5900 | b |
| Gene_7 | 72 hr | Laminarin + virus | 19.008 | 20.416 | 14.535 | 17.986 | 0.3070 | 0.1773 | ab |
| Gene_8 | 12 hr | H2O | 10.000 | 10.000 | 10.000 | 10.000 | 0.0000 | 0.0000 | a |
| Gene_8 | 12 hr | Laminarin | 0.8695 | 0.9166 | 10.042 | 0.9301 | 0.0684 | 0.0395 | a |
| Gene_8 | 12 hr | Virus | 0.9192 | 0.9582 | 0.9766 | 0.9513 | 0.0293 | 0.0169 | a |
| Gene_8 | 12 hr | Laminarin + virus | 0.9892 | 10.375 | 10.132 | 10.133 | 0.0242 | 0.0139 | a |
| Gene_8 | 24 hr | H2O | 10.000 | 10.000 | 10.000 | 10.000 | 0.0000 | 0.0000 | a |
| Gene_8 | 24 hr | Laminarin | 12.571 | 13.299 | 11.584 | 12.485 | 0.0861 | 0.0497 | a |
| Gene_8 | 24 hr | Virus | 0.8320 | 0.9801 | 11.014 | 0.9711 | 0.1349 | 0.0779 | a |
| Gene_8 | 24 hr | Laminarin + virus | 11.335 | 11.617 | 0.9361 | 10.771 | 0.1229 | 0.0710 | a |
| Gene_8 | 48 hr | H2O | 10.000 | 10.000 | 10.000 | 10.000 | 0.0000 | 0.0000 | a |
| Gene_8 | 48 hr | Laminarin | 12.258 | 0.9690 | 15.958 | 12.635 | 0.3151 | 0.1819 | a |
| Gene_8 | 48 hr | Virus | 10.712 | 10.798 | 13.813 | 11.774 | 0.1766 | 0.1020 | a |
| Gene_8 | 48 hr | Laminarin + virus | 13.157 | 0.7588 | 11.274 | 10.673 | 0.2833 | 0.1636 | a |
| Gene_8 | 72 hr | H2O | 10.000 | 10.000 | 10.000 | 10.000 | 0.0000 | 0.0000 | a |
| Gene_8 | 72 hr | Laminarin | 24.387 | 43.671 | 32.797 | 33.618 | 0.9668 | 0.5582 | a |
| Gene_8 | 72 hr | Virus | 16.300 | 16.356 | 15.680 | 16.112 | 0.0375 | 0.0217 | a |
| Gene_8 | 72 hr | Laminarin + virus | 48.220 | 49.687 | 22.018 | 39.975 | 15.569 | 0.8989 | a |
| Gene_9 | 12 hr | H2O | 10.000 | 10.000 | 10.000 | 10.000 | 0.0000 | 0.0000 | a |
| Gene_9 | 12 hr | Laminarin | 0.8527 | 0.8398 | 10.240 | 0.9055 | 0.1028 | 0.0594 | a |
| Gene_9 | 12 hr | Virus | 0.8548 | 0.8133 | 10.437 | 0.9039 | 0.1228 | 0.0709 | a |
| Gene_9 | 12 hr | Laminarin + virus | 0.9838 | 0.8556 | 10.797 | 0.9730 | 0.1124 | 0.0649 | a |
| Gene_9 | 24 hr | H2O | 10.000 | 10.000 | 10.000 | 10.000 | 0.0000 | 0.0000 | a |
| Gene_9 | 24 hr | Laminarin | 13.867 | 0.9303 | 10.466 | 11.212 | 0.2372 | 0.1369 | a |
| Gene_9 | 24 hr | Virus | 0.9580 | 13.849 | 0.9184 | 10.871 | 0.2587 | 0.1493 | a |
| Gene_9 | 24 hr | Laminarin + virus | 0.8394 | 10.098 | 11.565 | 10.019 | 0.1587 | 0.0916 | a |
| Gene_9 | 48 hr | H2O | 10.000 | 10.000 | 10.000 | 10.000 | 0.0000 | 0.0000 | a |
| Gene_9 | 48 hr | Laminarin | 16.481 | 15.436 | 13.847 | 15.255 | 0.1326 | 0.0766 | ab |
| Gene_9 | 48 hr | Virus | 15.027 | 16.392 | 21.260 | 17.560 | 0.3277 | 0.1892 | b |
| Gene_9 | 48 hr | Laminarin + virus | 15.088 | 17.637 | 13.474 | 15.400 | 0.2099 | 0.1212 | ab |
| Gene_9 | 72 hr | H2O | 10.000 | 10.000 | 10.000 | 10.000 | 0.0000 | 0.0000 | a |
| Gene_9 | 72 hr | Laminarin | 16.204 | 0.7923 | 18.354 | 14.160 | 0.5508 | 0.3180 | a |
| Gene_9 | 72 hr | Virus | 10.255 | 10.099 | 0.9752 | 10.035 | 0.0258 | 0.0149 | a |
| Gene_9 | 72 hr | Laminarin + virus | 0.9998 | 10.127 | 0.9578 | 0.9901 | 0.0287 | 0.0166 | a |
| Gene_10 | 12 hr | H2O | 10.000 | 10.000 | 10.000 | 10.000 | 0.0000 | 0.0000 | a |
| Gene_10 | 12 hr | Laminarin | 10.134 | 11.716 | 14.046 | 11.966 | 0.1968 | 0.1136 | a |
| Gene_10 | 12 hr | Virus | 12.765 | 13.547 | 11.548 | 12.620 | 0.1007 | 0.0582 | a |
| Gene_10 | 12 hr | Laminarin + virus | 11.314 | 15.111 | 14.571 | 13.665 | 0.2054 | 0.1186 | a |
| Gene_10 | 24 hr | H2O | 10.000 | 10.000 | 10.000 | 10.000 | 0.0000 | 0.0000 | a |
| Gene_10 | 24 hr | Laminarin | 13.673 | 15.462 | 14.026 | 14.387 | 0.0948 | 0.0547 | a |
| Gene_10 | 24 hr | Virus | 10.549 | 13.120 | 13.679 | 12.450 | 0.1669 | 0.0964 | a |
| Gene_10 | 24 hr | Laminarin + virus | 12.827 | 10.653 | 14.308 | 12.596 | 0.1839 | 0.1062 | a |
| Gene_10 | 48 hr | H2O | 10.000 | 10.000 | 10.000 | 10.000 | 0.0000 | 0.0000 | a |
| Gene_10 | 48 hr | Laminarin | 14.550 | 13.402 | 19.692 | 15.882 | 0.3349 | 0.1934 | a |
| Gene_10 | 48 hr | Virus | 0.8418 | 10.060 | 13.629 | 10.702 | 0.2664 | 0.1538 | a |
| Gene_10 | 48 hr | Laminarin + virus | 10.393 | 11.439 | 11.568 | 11.133 | 0.0644 | 0.0372 | a |
| Gene_10 | 72 hr | H2O | 10.000 | 10.000 | 10.000 | 10.000 | 0.0000 | 0.0000 | a |
| Gene_10 | 72 hr | Laminarin | 28.092 | 16.708 | 22.307 | 22.369 | 0.5692 | 0.3286 | a |
| Gene_10 | 72 hr | Virus | 15.579 | 22.088 | 17.467 | 18.378 | 0.3348 | 0.1933 | a |
| Gene_10 | 72 hr | Laminarin + virus | 10.994 | 19.941 | 16.529 | 15.821 | 0.4515 | 0.2607 | a |
| Gene_11 | 12 hr | H2O | 10.000 | 10.000 | 10.000 | 10.000 | 0.0000 | 0.0000 | a |
| Gene_11 | 12 hr | Laminarin | 0.9441 | 0.8925 | 11.302 | 0.9889 | 0.1250 | 0.0722 | a |
| Gene_11 | 12 hr | Virus | 10.548 | 0.9886 | 10.999 | 10.478 | 0.0560 | 0.0323 | a |
| Gene_11 | 12 hr | Laminarin + virus | 12.101 | 11.431 | 10.206 | 11.246 | 0.0961 | 0.0555 | a |
| Gene_11 | 24 hr | H2O | 10.000 | 10.000 | 10.000 | 10.000 | 0.0000 | 0.0000 | a |
| Gene_11 | 24 hr | Laminarin | 17.183 | 12.595 | 13.075 | 14.284 | 0.2522 | 0.1456 | a |
| Gene_11 | 24 hr | Virus | 12.194 | 11.235 | 10.512 | 11.314 | 0.0844 | 0.0487 | a |
| Gene_11 | 24 hr | Laminarin + virus | 13.862 | 11.424 | 11.236 | 12.174 | 0.1465 | 0.0846 | a |
| Gene_11 | 48 hr | H2O | 10.000 | 10.000 | 10.000 | 10.000 | 0.0000 | 0.0000 | a |
| Gene_11 | 48 hr | Laminarin | 34.926 | 33.905 | 24.218 | 31.016 | 0.5909 | 0.3412 | c |
| Gene_11 | 48 hr | Virus | 12.590 | 13.877 | 10.829 | 12.432 | 0.1530 | 0.0883 | ab |
| Gene_11 | 48 hr | Laminarin + virus | 28.058 | 20.254 | 34.812 | 27.708 | 0.7286 | 0.4206 | bc |
| Gene_11 | 72 hr | H2O | 10.000 | 10.000 | 10.000 | 10.000 | 0.0000 | 0.0000 | a |
| Gene_11 | 72 hr | Laminarin | 47.958 | 51.518 | 48.365 | 49.280 | 0.1948 | 0.1125 | b |
| Gene_11 | 72 hr | Virus | 12.075 | 13.414 | 13.319 | 12.936 | 0.0747 | 0.0431 | a |
| Gene_11 | 72 hr | Laminarin + virus | 80.672 | 76.025 | 53.346 | 70.014 | 14.621 | 0.8442 | b |
| Gene_12 | 12 hr | H2O | 10.000 | 10.000 | 10.000 | 10.000 | 0.0000 | 0.0000 | a |
| Gene_12 | 12 hr | Laminarin | 0.9616 | 11.039 | 10.693 | 10.449 | 0.0742 | 0.0428 | a |
| Gene_12 | 12 hr | Virus | 11.212 | 11.895 | 0.9477 | 10.861 | 0.1247 | 0.0720 | a |
| Gene_12 | 12 hr | Laminarin + virus | 10.097 | 0.9948 | 11.722 | 10.589 | 0.0984 | 0.0568 | a |
| Gene_12 | 24 hr | H2O | 10.000 | 10.000 | 10.000 | 10.000 | 0.0000 | 0.0000 | a |
| Gene_12 | 24 hr | Laminarin | 11.678 | 11.217 | 10.555 | 11.150 | 0.0564 | 0.0326 | a |
| Gene_12 | 24 hr | Virus | 10.143 | 12.364 | 10.635 | 11.047 | 0.1167 | 0.0674 | a |
| Gene_12 | 24 hr | Laminarin + virus | 0.9321 | 11.344 | 11.191 | 10.619 | 0.1127 | 0.0650 | a |
| Gene_12 | 48 hr | H2O | 10.000 | 10.000 | 10.000 | 10.000 | 0.0000 | 0.0000 | a |
| Gene_12 | 48 hr | Laminarin | 46.753 | 1.04.086 | 31.171 | 60.670 | 38.398 | 22.169 | a |
| Gene_12 | 48 hr | Virus | 0.5595 | 22.242 | 13.761 | 13.866 | 0.8324 | 0.4806 | a |
| Gene_12 | 48 hr | Laminarin + virus | 42.751 | 56.022 | 37.918 | 45.564 | 0.9374 | 0.5412 | a |
| Gene_12 | 72 hr | H2O | 10.000 | 10.000 | 10.000 | 10.000 | 0.0000 | 0.0000 | a |
| Gene_12 | 72 hr | Laminarin | 44.543 | 23.924 | 31.675 | 33.381 | 10.415 | 0.6013 | b |
| Gene_12 | 72 hr | Virus | 11.052 | 11.953 | 0.7904 | 10.303 | 0.2125 | 0.1227 | ab |
| Gene_12 | 72 hr | Laminarin + virus | 36.333 | 29.356 | 21.584 | 29.091 | 0.7378 | 0.4260 | ab |
| Gene_13 | 12 hr | H2O | 10.000 | 10.000 | 10.000 | 10.000 | 0.0000 | 0.0000 | a |
| Gene_13 | 12 hr | Laminarin | 0.9025 | 11.021 | 12.323 | 10.790 | 0.1661 | 0.0959 | a |
| Gene_13 | 12 hr | Virus | 11.788 | 12.230 | 10.116 | 11.378 | 0.1115 | 0.0644 | a |
| Gene_13 | 12 hr | Laminarin + virus | 13.339 | 10.934 | 11.013 | 11.762 | 0.1366 | 0.0789 | a |
| Gene_13 | 24 hr | H2O | 10.000 | 10.000 | 10.000 | 10.000 | 0.0000 | 0.0000 | a |
| Gene_13 | 24 hr | Laminarin | 13.750 | 14.763 | 13.300 | 13.938 | 0.0750 | 0.0433 | b |
| Gene_13 | 24 hr | Virus | 11.851 | 12.774 | 10.569 | 11.731 | 0.1107 | 0.0639 | ab |
| Gene_13 | 24 hr | Laminarin + virus | 13.085 | 11.438 | 12.395 | 12.306 | 0.0827 | 0.0477 | ab |
| Gene_13 | 48 hr | H2O | 10.000 | 10.000 | 10.000 | 10.000 | 0.0000 | 0.0000 | a |
| Gene_13 | 48 hr | Laminarin | 31.999 | 13.498 | 29.839 | 25.112 | 10.116 | 0.5841 | a |
| Gene_13 | 48 hr | Virus | 14.885 | 0.3825 | 0.8536 | 0.9082 | 0.5551 | 0.3205 | a |
| Gene_13 | 48 hr | Laminarin + virus | 25.291 | 0.8970 | 28.993 | 21.085 | 10.654 | 0.6151 | a |
| Gene_13 | 72 hr | H2O | 10.000 | 10.000 | 10.000 | 10.000 | 0.0000 | 0.0000 | a |
| Gene_13 | 72 hr | Laminarin | 45.838 | 96.757 | 93.370 | 78.655 | 28.471 | 16.438 | b |
| Gene_13 | 72 hr | Virus | 10.302 | 20.326 | 0.9999 | 13.542 | 0.5877 | 0.3393 | a |
| Gene_13 | 72 hr | Laminarin + virus | 53.674 | 42.556 | 49.901 | 48.710 | 0.5654 | 0.3264 | ab |
| Gene_14 | 12 hr | H2O | 10.000 | 10.000 | 10.000 | 10.000 | 0.0000 | 0.0000 | a |
| Gene_14 | 12 hr | Laminarin | 12.337 | 10.845 | 11.390 | 11.524 | 0.0755 | 0.0436 | a |
| Gene_14 | 12 hr | Virus | 0.9975 | 14.932 | 12.051 | 12.319 | 0.2489 | 0.1437 | a |
| Gene_14 | 12 hr | Laminarin + virus | 14.800 | 14.769 | 10.304 | 13.291 | 0.2587 | 0.1494 | a |
| Gene_14 | 24 hr | H2O | 10.000 | 10.000 | 10.000 | 10.000 | 0.0000 | 0.0000 | a |
| Gene_14 | 24 hr | Laminarin | 15.741 | 11.640 | 12.032 | 13.138 | 0.2263 | 0.1307 | a |
| Gene_14 | 24 hr | Virus | 0.9460 | 14.918 | 14.488 | 12.956 | 0.3035 | 0.1752 | a |
| Gene_14 | 24 hr | Laminarin + virus | 10.995 | 12.080 | 12.197 | 11.758 | 0.0663 | 0.0383 | a |
| Gene_14 | 48 hr | H2O | 10.000 | 10.000 | 10.000 | 10.000 | 0.0000 | 0.0000 | a |
| Gene_14 | 48 hr | Laminarin | 0.3028 | 0.7177 | 0.5353 | 0.5186 | 0.2080 | 0.1201 | a |
| Gene_14 | 48 hr | Virus | 0.6528 | 12.416 | 0.9372 | 0.9439 | 0.2944 | 0.1700 | a |
| Gene_14 | 48 hr | Laminarin + virus | 0.5979 | 0.3384 | 0.4902 | 0.4755 | 0.1304 | 0.0753 | a |
| Gene_14 | 72 hr | H2O | 10.000 | 10.000 | 10.000 | 10.000 | 0.0000 | 0.0000 | bc |
| Gene_14 | 72 hr | Laminarin | 0.2805 | 0.3706 | 0.3064 | 0.3192 | 0.0464 | 0.0268 | a |
| Gene_14 | 72 hr | Virus | 15.462 | 12.525 | 0.9925 | 12.637 | 0.2770 | 0.1599 | c |
| Gene_14 | 72 hr | Laminarin + virus | 0.5857 | 0.5072 | 0.2692 | 0.4540 | 0.1648 | 0.0952 | ab |
| Gene_15 | 12 hr | H2O | 10.000 | 10.000 | 10.000 | 10.000 | 0.0000 | 0.0000 | a |
| Gene_15 | 12 hr | Laminarin | 10.060 | 10.254 | 12.879 | 11.065 | 0.1575 | 0.0909 | a |
| Gene_15 | 12 hr | Virus | 13.362 | 16.199 | 11.923 | 13.828 | 0.2176 | 0.1256 | a |
| Gene_15 | 12 hr | Laminarin + virus | 12.002 | 16.131 | 13.764 | 13.966 | 0.2072 | 0.1196 | a |
| Gene_15 | 24 hr | H2O | 10.000 | 10.000 | 10.000 | 10.000 | 0.0000 | 0.0000 | a |
| Gene_15 | 24 hr | Laminarin | 13.129 | 21.363 | 13.388 | 15.960 | 0.4681 | 0.2703 | a |
| Gene_15 | 24 hr | Virus | 11.647 | 13.931 | 10.641 | 12.073 | 0.1686 | 0.0973 | a |
| Gene_15 | 24 hr | Laminarin + virus | 15.392 | 14.812 | 12.195 | 14.133 | 0.1703 | 0.0983 | a |
| Gene_15 | 48 hr | H2O | 10.000 | 10.000 | 10.000 | 10.000 | 0.0000 | 0.0000 | a |
| Gene_15 | 48 hr | Laminarin | 32.421 | 0.8602 | 19.066 | 20.030 | 11.939 | 0.6893 | a |
| Gene_15 | 48 hr | Virus | 11.066 | 0.7972 | 11.645 | 10.228 | 0.1975 | 0.1140 | a |
| Gene_15 | 48 hr | Laminarin + virus | 16.304 | 17.934 | 18.959 | 17.732 | 0.1339 | 0.0773 | a |
| Gene_15 | 72 hr | H2O | 10.000 | 10.000 | 10.000 | 10.000 | 0.0000 | 0.0000 | a |
| Gene_15 | 72 hr | Laminarin | 49.281 | 36.255 | 43.025 | 42.853 | 0.6515 | 0.3761 | b |
| Gene_15 | 72 hr | Virus | 0.8287 | 13.318 | 10.022 | 10.542 | 0.2555 | 0.1475 | a |
| Gene_15 | 72 hr | Laminarin + virus | 22.660 | 30.703 | 44.821 | 32.728 | 11.218 | 0.6477 | ab |

1. A method of increasing plant immunity, plant defenses and tolerance to insect and fungal vectored pathogens, increasing plant disease or pest resistance, protecting plants from disease, or improving plant disease management, by modulating gene expression of plant defense genes, said method comprising applying a composition comprising laminarin to said plant.

2. The method according to claim 1, wherein said pathogen is a virus causing the disease.

3. The method according to claim 2, wherein said virus is selected from the group consisting of a virus of the genus Orthotospovirus, a virus of the genus Cucumovirus, a virus of the genus Potyvirus, a virus of the genus Caulimovirus, and a virus of the genus Tobamovirus.

4. The method according to claim 3, wherein said virus is Tomato Spotted Wilt Virus (TSWV), Cucumber Mosaic Virus (CMV), Potato Virus Y (PVY), Cauliflower Mosaic Virus (CaMV) or Tobacco Mosaic Virus (TMV).

5. The method according to claim 1, comprising applying the laminarin to a locus of said plant.

6. The method according to claim 1, wherein at least one plant defense gene is modulated.

7. The method according to claim 6, wherein said at least one plant defense gene is switched on, overexpressed or upregulated.

8. The method according to claim 6, wherein said at least one plant defense gene is switched off, under-expressed or downregulated.

9. The method according to claim 1, wherein expression of one or more of the following genes is modulated: E3 Ubiquitin-protein ligase march3, Nbs-lrr resistance protein, AP2-like-ethylene-responsive transcription factor, Avr/Cf9 rapidly elicited protein 75, 1-aminocryclopropane-1-carboxylate oxidase-like protein, Hypersensitive response assisting protein, Nac transcription factor 29-like, Subtilisin-like protease-like, Tropinone reductase homolog at 1g07440-like, Pathogenesis-related protein P4, PR protein, AY093595.1, Regulator of gene silencing, and Pti6.

10. The method according to claim 1, wherein one or more of the following genes are overexpressed or upregulated: E3 Ubiquitin-protein ligase march3, Nbs-lrr resistance protein, AP2-like-ethylene-responsive transcription factor, Avr/Cf9 rapidly elicited protein 75, 1-aminocryclopropane-1-carboxylate oxidase-like protein, Hypersensitive response assisting protein, Nac transcription factor 29-like, Tropinone reductase homolog at 1g07440-like, Pathogenesis-related protein P4, PR protein, AY093595.1, and Pti6.

11. The method according to claim 1, wherein Regulator of gene silencing is underexpressed or down regulated.

12. A method of controlling a viral disease or a disease caused by insect and fungal vectored pathogens in a plant, comprising applying a composition comprising laminarin to said plant, wherein the laminarin modulates gene expression of plant defense genes in said plant.

13. (canceled)

14. (canceled)

15. (canceled)

16. A method of characterizing gene expression of plant defense genes effected by applying a composition comprising laminarin to the said plant, comprising: a) extraction of genetic material from the subject plant; b) optionally, amplification of the said genetic material; and c) analysis of the expression levels of the said genetic material; wherein an increased level of expression of a putative plant defense gene compared to a control indicates that laminarin is eliciting overexpression or upregulation of said gene, or a decreased level of expression of a putative plant defense gene compared to a control indicates that laminarin is eliciting underexpression or downregulation of said gene.


SEQUENCE LISTING (30 sequences)

SEQ ID NO: 1 - DNA, 20 bp, Artificial Sequence
  primer
tgacgaagtcaggacaggaa

SEQ ID NO: 2 - DNA, 20 bp, Artificial Sequence
  primer
ctgcatcttctttgccactg

SEQ ID NO: 3 - DNA, 24 bp, Artificial Sequence
  primer
gaaagagaaatgctggtgaggagt

SEQ ID NO: 4 - DNA, 24 bp, Artificial Sequence
  primer
ctcctccattcatcacctctaaca

SEQ ID NO: 5 - DNA, 24 bp, Artificial Sequence
  primer
actattattctcccctcgcgtctc

SEQ ID NO: 6 - DNA, 24 bp, Artificial Sequence
  primer
gtaagcaccgagaacccaatgact

SEQ ID NO: 7 - DNA, 22 bp, Artificial Sequence
  primer
ccgcaatcaaatgtaatggaag

SEQ ID NO: 8 - DNA, 24 bp, Artificial Sequence
  primer
aactatcggtgaaccagatttgtc

SEQ ID NO: 9 - DNA, 23 bp, Artificial Sequence
  primer
aggaggggggttgaagaagagta

SEQ ID NO: 10 - DNA, 25 bp, Artificial Sequence
  primer
aaacccttctttcttcttttttcca

SEQ ID NO: 11 - DNA, 24 bp, Artificial Sequence
  primer
atgagcaacgataggtacaagagc

SEQ ID NO: 12 - DNA, 24 bp, Artificial Sequence
  primer
tgggcactgacactctgtttctac

SEQ ID NO: 13 - DNA, 20 bp, Artificial Sequence
  primer
aaattcagcagcaccaccag

SEQ ID NO: 14 - DNA, 20 bp, Artificial Sequence
  primer
tcctctttgatctgctggcc

SEQ ID NO: 15 - DNA, 20 bp, Artificial Sequence
  primer
acaagttcgatccctgggta

SEQ ID NO: 16 - DNA, 20 bp, Artificial Sequence
  primer
cacagctgctctatttggcc

SEQ ID NO: 17 - DNA, 20 bp, Artificial Sequence
  primer
tttggcgaatgatcccactg

SEQ ID NO: 18 - DNA, 20 bp, Artificial Sequence
  primer
acacctttggcgaaattccc

SEQ ID NO: 19 - DNA, 20 bp, Artificial Sequence
  primer
cacttgatggtttggctgct

SEQ ID NO: 20 - DNA, 20 bp, Artificial Sequence
  primer
cttttgcaccaagtccagct

SEQ ID NO: 21 - DNA, 20 bp, Artificial Sequence
  primer
tcttgcggttcacaacgatg

SEQ ID NO: 22 - DNA, 20 bp, Artificial Sequence
  primer
ccgctcttgagttggcatag

SEQ ID NO: 23 - DNA, 20 bp, Artificial Sequence
  primer
caagtagtctggcgcaactc

SEQ ID NO: 24 - DNA, 20 bp, Artificial Sequence
  primer
tccgatccagttgcctacag

SEQ ID NO: 25 - DNA, 20 bp, Artificial Sequence
  primer
ggccaaacatgggtcatcaa

SEQ ID NO: 26 - DNA, 20 bp, Artificial Sequence
  primer
taggaccccaccacaatcac

SEQ ID NO: 27 - DNA, 20 bp, Artificial Sequence
  primer
aggcggtaggaggtgagata

SEQ ID NO: 28 - DNA, 20 bp, Artificial Sequence
  primer
cctcaaaccctaacaacccg

SEQ ID NO: 29 - DNA, 20 bp, Artificial Sequence
  primer
aggcggtaggaggtgagata

SEQ ID NO: 30 - DNA, 20 bp, Artificial Sequence
  primer
cctcaaaccctaacaacccg